(19) United States
(12) Patent Application Publication
(10) Pub. No.: US 2023/0218160 A1
(43) Pub. Date: Jul. 13, 2023

(54) SELF SERVICE REFRACTION DEVICE AND METHOD

(71) Applicant: Shui T LAI, Windermere, FL (US)

(72) Inventor: Shui T LAI, Windermere, FL (US)

(21) Appl. No.: 17/800,532

(22) PCT Filed: Aug. 27, 2020
(86) PCT No.: PCT/US20/48304

§ 371 Date: Aug. 17, 2022

Related U.S. Application Data
(63) Continuation-in-part of application No. PCT/US2020/018712, filed on Feb. 18, 2020.

(51) Int. Cl. A61B 3/032; A61B 3/14

(52) U.S. Cl. CPC A61B 3/032; A61B 3/14

(57) ABSTRACT

A self service refraction instrument includes an adjustable optical assembly coupled within an instrument housing between an eyepiece and a viewing target display. A defocus correcting component includes a pair of adjustably spaced apart spherical lens elements. An astigmatism correcting component includes a pair of cylindrical lens elements that are synchronously rotatable in opposite directions for adjusting cylinder power and in a same direction for adjusting axis angle. A decision module informed by visual acuity scores for an autorefraction Rx or an old prescription Rx of a test subject, the test subject's communicated choices among presented options of corrective optics prescriptions and a refraction rules database that decides to next test sphere, axis or cylinder, at a specific step size of increments, or to test visual acuity, or to end a test.

### PRIORITY AND RELATED APPLICATIONS

This application is a continuation in part (CIP) of PCT/US20/18712, filed Feb. 18, 2020, which claims the benefit of priority to U.S. provisional patent application No. 62/806,911, filed Feb. 18, 2019. Each of these priority patent applications is incorporated by reference.

This application is related to U.S. Pat. Nos. 10,383,512, 10,194,796, 10,194,794, 9,955,867, 9,743,829, 9,730,578, 9,408,533, 9,320,426, 9,247,871, 8,967,801, 8,950,865, 8,790,104, 8,753,551, 8,684,527, 8,636,359, 8,632,184, 8,632,183, 8,409,177, 8,388,137, 8,366,274, 8,262,220, 8,113,658, 8,066,359, 8,033,664, 7,954,950, 7,909,461, 7,824,033, 7,748,844, 7,726,811, 7,699,471, 7,695,134, 7,490,940, 7,461,938, 7,425,067, 7,420,743, 7,293,871, 7,234,810, 7,220,255, 7,114,808, 7,114,415, 6,761,454, 6,706,036, 6,325,792, 6,210,401, and 5,549,632, which are incorporated by reference.

### BACKGROUND

Subjective refraction is a procedure that remains one of the most challenging to train a technician to produce a consistent and accurate outcome. Auto refraction can be helpful to provide a starting point. However, subjective refraction remains the gold standard. No conventional, existing auto refractor can serve as a suitable replacement for a subjective refraction eye exam in terms of accuracy. To be proficient in subjective refraction requires extensive training, and the learning curve is very steep. It can take months, or even years before a refraction technician may master the technique of subjective refraction.

It is desired to provide a self service refraction instrument and method. Thereby, a patient can perform the measurement, either alone or with the supervision of a minimally trained technician at relatively low cost. Ideally, a procedure that can be entirely self-service without an expert operator present for at least a significant proportion of the testing time is greatly desired and would provide tremendous cost advantage. An entirely self-service subjective refraction instrument and procedure would provide even more cost saving by not needing the presence of any person other than the test subject herself or himself to run the refraction test. With such desired self service refraction instrument and method, a test subject or eye care patient could perform the measurement at a self service kiosk during which time the physician or optometrist would not need to be present, thus saving the test subject money. The desired self service eye testing solution would not require long term training or high cost, available during the duration of the self service refraction procedure. Such self service or minimal supervision refraction test would increase its usability, and provides substantial cost saving. The operation of the desired self service refraction device would also not be limited by the work hours of an operator of a conventional refraction eye examination instrument. The conventional operator-dependent refraction instrument may be idled due to sick days, vacation, leaves, and holidays.

### BRIEF DESCRIPTION OF THE DRAWINGS

FIG. 1 illustrates a workflow of a self-service refraction test by decision module in accordance with an example embodiment.

FIG. 2 illustrates an example of an auto refraction process preceding a subjective refraction eye examination in accordance with an example embodiment.

FIG. 3A illustrates an example of a subjective refraction process that utilizes a self service refraction kiosk system that includes an adjustable optical assembly that is controlled by a processor programmed by on-board decision module in accordance with an example embodiment.

FIG. 3B illustrates another example of a subjective refraction process that utilizes a self service refraction kiosk system that includes an adjustable optical assembly that is controlled by a processor programmed by on-board decision module in accordance with an example embodiment.

FIG. 3C illustrates a further example of a subjective refraction process that utilizes a self service refraction kiosk system that includes an adjustable optical assembly that is controlled by a processor programmed by on-board decision module in accordance with an example embodiment.

FIG. 3D illustrates another example of a subjective refraction process that utilizes a self service refraction kiosk system that includes an adjustable optical assembly that is controlled by a processor programmed by on-board decision module in accordance with an example embodiment.

FIG. 4A schematically illustrates a self-service refraction by decision module apparatus in block form in accordance with an example embodiment with arrows indicating directions of information flow between components of the apparatus and to and from a test subject who is self-administering an eye test using the apparatus.

FIG. 4B illustrates an example embodiment of a self-service refraction apparatus including a decision module configured in accordance with a rules database to instruct a processor in accordance with certain decisions regarding next steps for presenting a viewing target to a test subject that includes an optical assembly configured with an adjustable sphere power and with an adjustable astigmatism power, and also in an example embodiment, with an adjustable astigmatism axis angle.

FIG. 4C illustrates a self-service refraction apparatus including a decision module in accordance with an example embodiment, wherein the decision module is informed or programmed by, or is configured in accordance with, a rules database to instruct a processor in accordance with certain decisions regarding next steps in a process for generating a corrective optical prescription for a test subject, including presenting a viewing target to the test subject through a corrective optical assembly configured with an adjustable sphere power and with an adjustable astigmatism power, and also in an example embodiment, with an adjustable astigmatism axis angle.

FIG. 4D illustrates a self-service refraction apparatus including a decision module in accordance with another example embodiment, wherein the decision module is informed by or is programmed by, or is configured in accordance with, a rules database, as well as information communicated from the test subject including preferences or choices among presented options of corrective optics and answers to questions formulated by the decision module about how images of viewing targets appear to the test subject to instruct a processor in accordance with certain decisions regarding next steps in a process for generating a corrective optical prescription for the test subject, including presenting a viewing target to the test subject through a corrective optical assembly configured with an adjustable sphere power and with an adjustable astigmatism power, and also in an example embodiment, with an adjustable astigmatism axis angle.

FIG. 4E illustrates a self-service refraction by decision module apparatus in accordance with an example embodiment, wherein the decision module may be informed by a rules database from refraction experts, physicians or the AMA or other industry standard setting organization and/or by an empirical database of machine learning, e.g., using a neural network, and by input from a test subject who is self-administering a refraction eye test using the apparatus.

FIG. 5A is a split flow chart illustrating two examples of self-service refraction by decision module processes in accordance with example embodiments, including a first process of the decision module selecting a first subset of optics to test when the test subject has an astigmatism issue and a second process of the decision module efficiently selecting a second, smaller subset of optics to test when the test subject does not have an astigmatism issue.

FIG. 5B is a flow chart illustrating an example of a first part of a method for identifying an axis angle of a test subject's astigmatism including a specific example ordering of steps and step sizes determined by the decision module in accordance with an example embodiment.

FIG. 5C is a flow chart illustrating an example of a second part of a method for identifying an axis angle of a test subject's astigmatism including a specific example ordering of steps and step sizes determined by the decision module in accordance with an example embodiment.

FIG. 6 illustrates an example method for determining an astigmatism power of a test subject including a specific example ordering of steps and step sizes determined by the decision module in accordance with an example embodiment.

FIGS. 7A-7G illustrate several example embodiments of viewing targets in accordance with certain embodiments either as determined by the decision module to be presented to a test subject before or after an adjustment of optical power and/or as they may appear to a test subject with uncorrected vision imperfections.

FIGS. 7H-7N illustrate several example embodiments of viewing targets in accordance with certain embodiments either as determined by the decision module to be presented to a test subject before or after an adjustment of optical power and/or as they may appear to a test subject with uncorrected vision imperfections.

FIG. 8A illustrates a point light source with a small line through it either as a viewing target for testing and correcting sphere, cylinder and axis for a test subject operating a self-service refraction instrument in accordance with an example embodiment, or representing a linear image appearing to a patient as adjusted from an initially blurry image of the point light source in accordance with an example embodiment.

FIG. 8B illustrates an example of a viewing target including a pair of crossed lines forming into an “X” shape and formed by spaced apart point light sources in accordance with an example embodiment.

FIG. 8C illustrates an example of an image of the viewing target of FIG. 8B as viewed by a test subject with uncorrected sphere and astigmatism eye aberrations, wherein the image appears as a series of small blurry line images arranged into the pair of crossed larger lines in the “X” shape of the viewing target in accordance with an example embodiment.

FIG. 8D illustrates an example of an image of the viewing target of FIG. 8B as viewed by a test subject with uncorrected astigmatism, which could be an image viewed by the same test subject as FIG. 8C with corrected sphere and partially corrected astigmatism in accordance with an example embodiment.

FIG. 8E illustrates an example of an image of the viewing target of FIG. 8B as viewed by a test subject with uncorrected sphere and astigmatism eye aberrations, or the image of FIG. 8E may be corrected for axis and partially corrected for astigmatism compared with the image of FIG. 8F which may be an image by the same test subject with uncorrected sphere, astigmatism and axis eye aberrations in accordance with an example embodiment.

FIG. 8F illustrates an example of an image of the viewing target of FIG. 8B as viewed by a test subject with uncorrected sphere, astigmatism and axis eye aberrations in accordance with an example embodiment, wherein FIG. 8E may be the image seen by the same test subject as corrected for axis and partially corrected for astigmatism, and FIG. 8D is the image seen by the same test subject as corrected for axis and sphere and partially for astigmatism, and FIG. 8B is an image entirely corrected for sphere, cylinder and axis, by using a self-service refraction apparatus and method in accordance with an example embodiment, which may have started off with the test subject seeing the viewing target as any of FIG. 8C, 8D, 8E or 8F.

FIG. 9 is a flow chart illustrating a method of self-service subjective refraction by decision module including multiple binary decision points at which the decision module instructs the processor to take certain next steps based on its decisions informed by current test data and a refraction rules database in accordance with an example embodiment.

FIG. 10A illustrates an initially blurry image of a point light source in accordance with an example embodiment.

FIG. 10B illustrates the blurry image of FIG. 10A converging to a more linear image in accordance with an example embodiment.

FIG. 10C illustrates a most substantially focused linear image indicated by a patient starting with the image of FIG. 10B, in accordance with an example embodiment.

FIG. 10D illustrates the blurred linear image of FIG. 10B being reduced in its long dimension and collapsing to form a blurry round or slightly oblong shape, in accordance with an example embodiment.

FIG. 10E illustrates an example of a most symmetric shape indicated by a patient starting with the image of FIG. 10D, in accordance with an example embodiment.

FIG. 10F illustrates another example of a most substantially focused linear image indicated by the patient starting with the image of FIG. 10D, in accordance with an example embodiment.

FIG. 10G illustrates the image of FIG. 10F corrected in accordance with an embodiment.

FIG. 11 illustrates an eye exam patient or test subject and a self-service refraction device positioned for testing the test subject's eyes and a head set being worn by the test subject that includes an electronic level sensor configured to communicate to the test subject and/or to an on board processor of the self-service refraction device or a separate computer or mobile electronic device heat tilt angle data to inform a decision module in determining whether to accept, amend or reject certain autorefraction data and/or to provide signal notifications whenever the test subject's head tilt angle exceeds one or more threshold values in accordance with an example embodiment.

FIG. 12 schematically illustrates an example embodiment of an optical assembly, which includes an optional alternative embodiment to a decision module-informed, and processor-controlled phoropter, including a defocus corrector assembly (DCA), an astigmatism corrector assembly (ACA), a waveplate assembly and a viewing target, configured for a self-service subjective refraction apparatus in accordance with an example embodiment.

FIG. 13 schematically illustrates an optical instrument outer housing configured to stably contain an optical assembly for a self-service subjective refraction apparatus in accordance with an example embodiment.

### DETAILED DESCRIPTION OF THE EMBODIMENTS

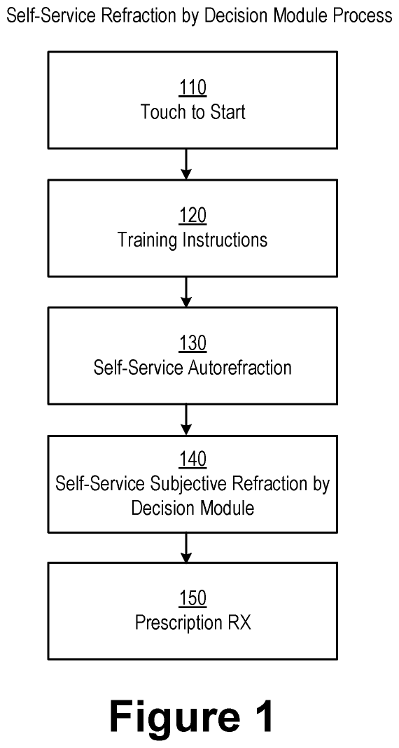

A self service refraction process in accordance with an example embodiment may be described with reference to FIG. 1 as follows:

A user of the service (from here on, the term of a patient, a test subject, a customer or a user of the service may be used interchangeably throughout the rest of the disclosure) who desires to have his or her vision improved by correcting refractive errors may approach a self service refraction instrument configured in accordance with an example embodiment.

A touch screen connecting to a computer for example, displays a message: “Touch to Start” as shown in 110 in FIG. 1. A test subject, a customer, a user or a patient walks up to the instrument and touches the screen in this example. A face detection process may alternatively be used to wake up or start the self-service refraction apparatus, and the specific test subject may be identified with face recognition unless he or she is new to the system, or a card may be inserted into a card reader, or a start button may be actuated at 110 in FIG. 1 in other example embodiments to start a self-service refraction procedure.

A training or introduction process may be displayed to the test subject with sound from speakers or may be provided as an option to new users which may be skipped by experienced users of the apparatus, and certain instructions may be provided, at 120 in FIG. 1. For example, a training video may be presented detailing how the entire refraction process may play out, and/or multiple optional video clips may be available to the test subject who may become thereby familiarized with the process. A brief tour of the main component sub-routines of the process may be provided to the test subject.

A training video may be displayed to the test subject at 120 in FIG. 1 which may provide instructions in a step by step manner as it may show how to perform a self-service auto refraction at 130 in FIG. 1 and followed by self-service subjective refraction at 140 in accordance with the example embodiment illustrated at FIG. 1. The training instructions 120 may include details of how to align the eye to the self-service refraction instrument, and/or how to position the eye to get the best results from an auto refraction component of the process as indicated at 130 in FIG. 1. A head tilt angle monitoring head gear or a face detection software with tilt, pose and/or portrait detection may be used to assist the test subject to maintain the correct posture for an autorefraction test. The training instructions 120 may also explain how a self-service subjective refraction component of the process as indicated at 140 in FIG. 1 may be performed, and how the customer should provide the kinds of responses that the apparatus will expect to receive from the test subject upon prompting by the computer-controlled interface of the self-service refraction apparatus, which may include computer-generated voice commands and/or pre-recorded human voice instructions, on screen text or image prompts and/or touch vibration prompts or communications. A self-service refraction apparatus in accordance with an example embodiment illustrated by FIG. 1 is configured to generate a corrective optical prescription 150 for the test subject that will enhance the visual acuity of the test subject.

### Human Interface Devices

A self-service refraction instrument in accordance with an example embodiment includes a computer processor or similar electronic device. The device is capable of receiving input from various human interface devices, such as a mouse, keyboard, touch screen, audio input, camera images, recognizing the presence of a patient in the vicinity, for example, and to perform measurements from the capture images, and so on.

### Self Service Auto Refraction

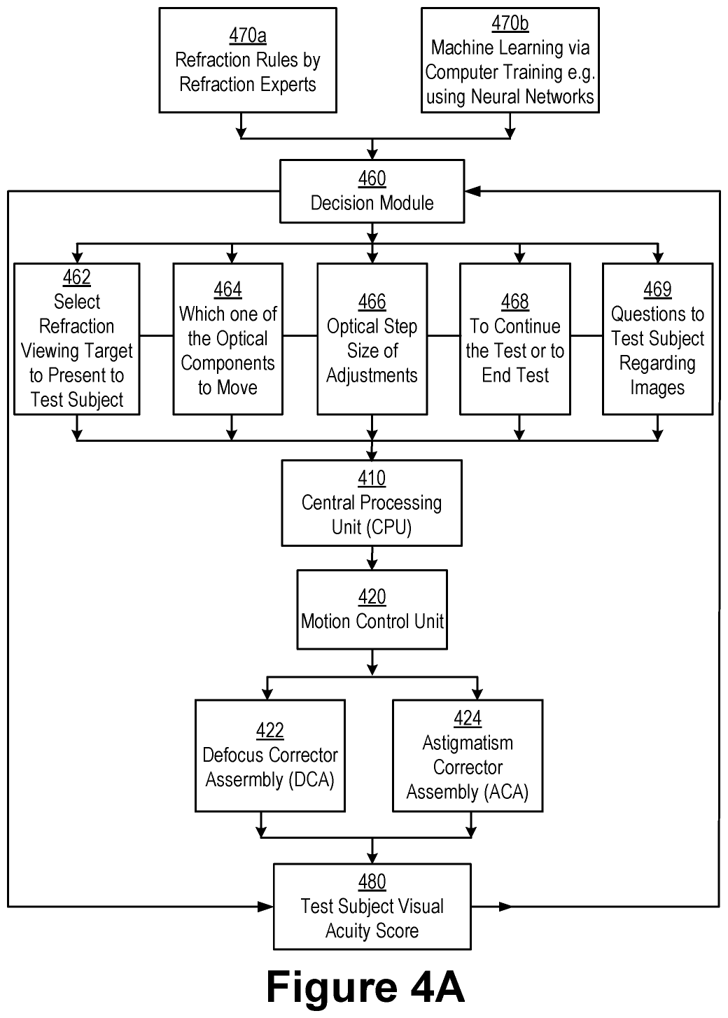

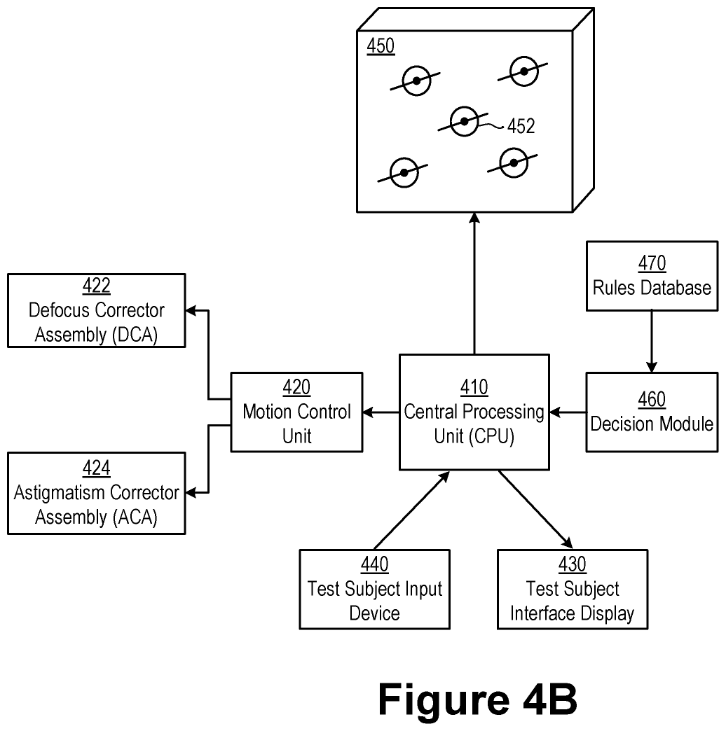

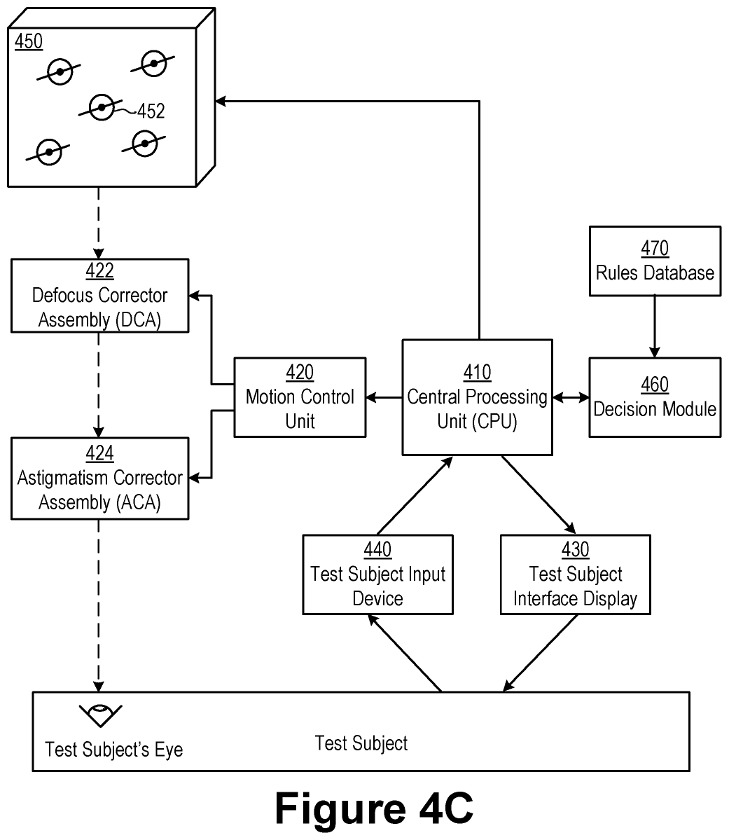

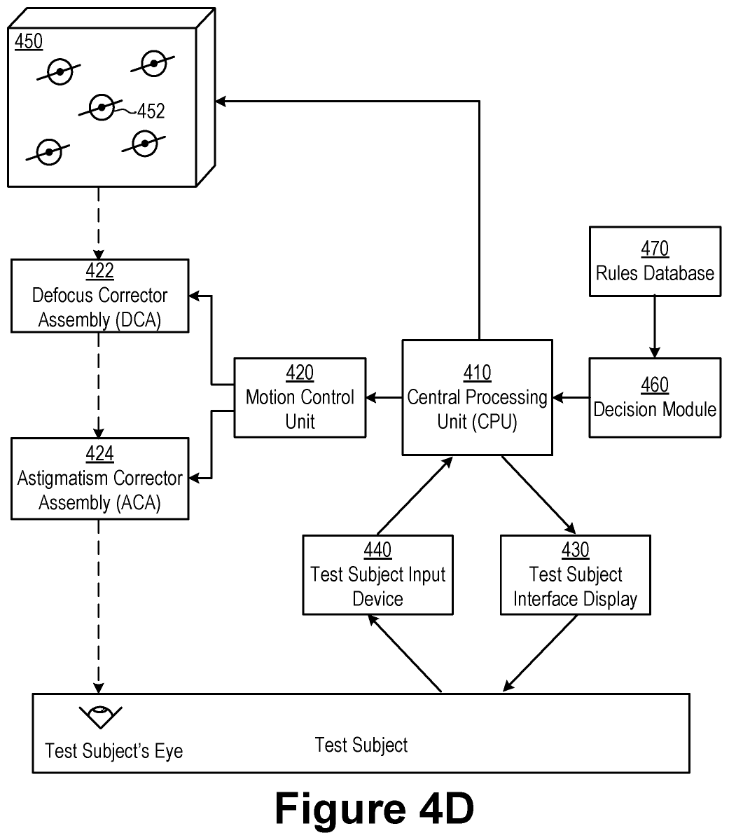

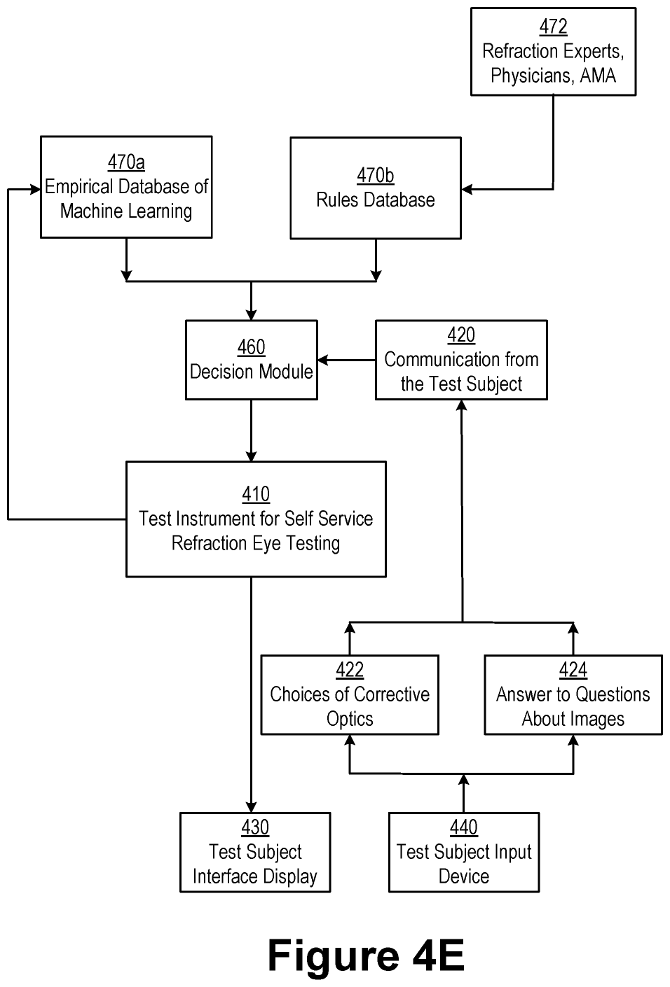

As shown in 130 in the example embodiment illustrated at FIG. 1, auto refraction may be performed after the training instructions in the form of video or other forms. One advantage of performing the auto refraction component 130 of the overall self-service refraction eye test is to obtain an efficient starting point for a subjective refraction component 140 of the test. A corrective optical prescription Rx may be generated at 150 in FIG. 1. One may assume that the initial prescription from auto refraction is closer to the final Rx of the user than starting from nothing. That is helpful to shorten the refraction time and confusion when performing the subsequent subjective refraction component 140 of the test. When an old eye prescription Rx is available as well as a self-service autorefraction Rx in accordance with an example embodiment, the decision module 460 of FIG. 4 may decide to check a visual acuity of the test subject for both the old prescription Rx and the autorefraction Rx, and may decide to start the self-service subjective refraction at the prescription corresponding to the higher visual acuity test score.

In one example embodiment, the self-service refraction instrument may include a part that permits the test subject to perform a self-service auto refraction eye test. Examples of a working auto refractor mechanism include a Nidek refractor model Tono Ref II and a Topcon auto refractor model KR-8000. The auto refraction 130 component of the test may use a wave front aberrometer device. The details of example wavefront optics and instrument construction have been disclosed in published PCT patent application number WO03/034909A2), which is incorporated by reference.

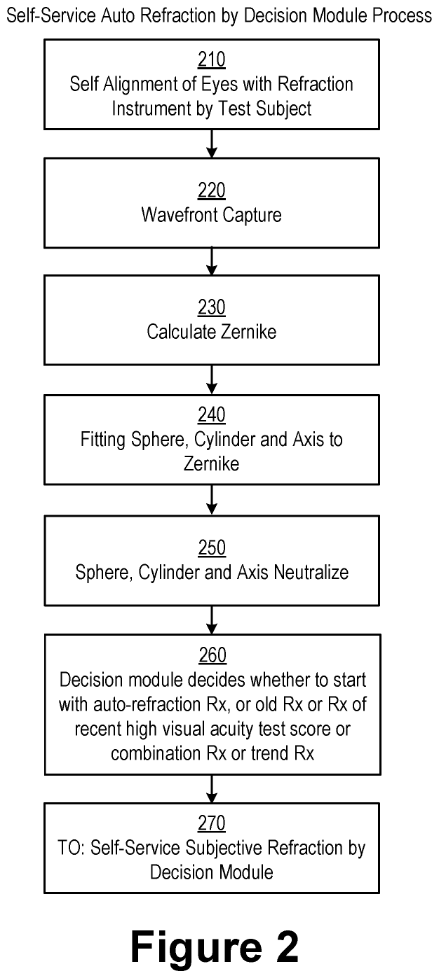

Referring now to an example auto refraction process illustrated at FIG. 2, the user's eye is to be aligned to the instrument at 210. The alignment of the eye of the test subject with the auto refraction device at 210 may be accomplished through watching a training video prior to the start of the refraction. Also voice guidance generated by the decision module 460 and processor 410 or computer 410 may be provided to instruct the user to place her eye or eyes within a certain region by moving the eye or eyes side to side, or up and down, or tilting to the left or to the right, around the eye piece of the instrument. Further, in 210, an image of the eye or eyes may be shown in a monitor such that the user may see her own eye pupil position. By providing a square or a circle to mark the ideal location for the eye or for each of the eyes, on a monitor, the test subject may be informed and may understand from a voice announcement that her pupil should be positioned inside a square, or a circle, or a generic image of an eye, as much centered as possible to get a good auto refraction image capture.

In an example embodiment, the test subject may perform self alignment of the test subject's eye or eyes with an auto refraction instrument, which may include:

a. An eye piece of the instrument;

b. A light source, projecting light into the eye, wherein light is reflected at the retina, and exits the pupil forming a lighted glow of the pupil;

c. A camera capturing the image of the user's pupil;

d. A monitor displaying the pupil image, wherein the user sees the pupil on the monitor;

e. A marker comprising a drawing of the boundary of a region, in which the intended eye location is within that region inside the marker's boundary, wherein the boundary can be in the shape of a square, a circle or an oval or ellipse or in the shape of a generic eye, wherein the center of the region defined by the boundary may be the intended location for the eye, and/or a pupil image may be overlapped with the pupil of the user when the pupil of the user is aligned.

The test subject or user of the self-service refraction instrument moves her eye to the center of a target boundary thereby aligning the eye to the intended position of the eye for an auto refraction image capture. Operation of the auto-refraction instrument and performance of an autorefraction eye test, and specifically alignment of the eyes of the user may include head tilt angle monitoring and feedback signals or other electronic or mechanical level guidance as may be described in further detail hereinbelow. Further examples include mechanical head stabilizing components such as a chin rest or forehead rest or other wearable head gear that may be mechanically coupled for stabilization relative to the auto refraction instrument or that may include an electronic level component that monitors head tilt angle and provides feedback to the user or an eye care technician regarding user head tilt angle before and/or during an auto refraction test.

Once the pupil positioning is done, the user may initiate a wave front image capture 220 as in the illustrative example embodiment of FIG. 2. Details of the construction of a wave front aberrometer in accordance with an example embodiment had been described previously in application WO03/034909A2, which is incorporated by reference. The wave front image of the eye is then analyzed. In one embodiment, the image may be decomposed into Zernike components 230. Using a method of mapping 240, the Zernike profile is best fit to match one set of values in the sphere, cylinder and axis. These aberrations of sphere, cylinder and axis may be neutralized with corrective optics 250 which may be utilized by the decision module 460 as a starting corrective optical assembly 260 for a self-service subjective refraction eye test by decision module 270.

In one embodiment, eye image capture is automatically triggered when the eye is inside the region defined by the marker. Certain eye image data may be gathered from analysis of the captured image of the test subject's eye including wavefront data of the eye, the pupil size, and the user's papillary distance. In certain embodiments, the optical path length and head tilt angle may be measured and utilized for instrument calibration and/or image data editing or processing.

In one embodiment, a method of converting a wavefront image to an auto refraction prescription may include the following steps:

a. Capturing a wavefront profile from a test subject using a self-service refraction apparatus at 220 in FIG. 2, using a camera, wherein the wavefront profile may be captured in the form of a wavefront image;

b. Decomposing the wavefront profile into Zernike components at 230 in FIG. 2 that represent the wavefront profile that is represented in the wavefront image;

c. Calculating a best fit that matches the decomposed wavefront profile to a set of values in sphere, cylinder and axis at 240 in FIG. 2, forming a correction wavefront profile, wherein the combination of the captured wavefront profile and the calculated correction profile results in improvement of visual acuity when comparing to the visual acuity of the captured wavefront profile subtracting off its second order terms.

The desired outcome is to identify the best sphere, cylinder and axis values 250, that provide the best fit to the eye's Zernike profile. These new second order terms from the calculation are the wavefront auto refraction correction terms that the decision module 460 may decide to use as a starting prescription of a corrective optics 260 for a self-service subjective refraction process 270 by applying these values to the corresponding optical components for sphere 422, and astigmatism 444 in the optical assembly that is used in the self-service subjective refraction test 270. A process in accordance with certain embodiments may include generating a correction wavefront profile based on measured aberrations of the eyes of a user and/or test subject using a self service refraction device in accordance with an example embodiment.

As illustrated in FIG. 2, a method of enhancing a visual acuity of a test subject's eyes which have certain specific, uncorrected aberrations, may include:

a. aligning test subject's eyes with an autorefraction instrument 210

b. measuring a wavefront image 220;

c. generating a wavefront profile 230;

d. calculating a Zernike profile 240;

e. calculating sphere, cylinder and axis values 250; and

f. generating a correction wavefront profile as a starting point 260 for a subjective refraction process 270.

### Self-Service Subjective Refraction by Decision Module

A self service subjective refraction eye examination by decision module in accordance with various example embodiments may take into account various test subject responses to questions involving comparative choices of corrective optics and the clarity, intensity and/or geometry of viewing targets observed through those choices of corrective optics. In a self-service subjective refraction eye examination by decision module in accordance with an example embodiment, a patient (the customer, the test subject or the user of the self-service refraction instrument, apparatus, device, or kiosk) has to pick among presented corrective vision options, for example, by indicating which option appears to present a viewing target with better clarity or better focus.

The viewing target may include one or more points, lines, letters and/or universal symbols, or a still image or scene, or a video sequence, or a live view or combinations thereof. The apparatus may provide a voice prompt, telling the patient what type of response is expected from her or him. The patient can use her voice, or another human user interface device, such as a mouse, a joystick, or a camera detection of hand gestures, or body movements to indicate choices, answers or picks, which will be received as input to a program running on the self-service refraction apparatus in accordance with an example embodiment that includes a processor-controlled, self service refraction test device that includes a decision module 460.

In an example situation during a self service refraction eye test, the patient may be asked by digital voice generation or text on a display screen, “which one is more clear, choice 1, or choice 2?” The patient may reply by saying into a microphone: “2.”, or he or she may use a mouse to roll the wheel up for answering “1” or roll down to indicate the answer is“2”, or by pushing a joystick up for “1” and pushing the joystick down for “2” or by tapping a touch screen or touch pad, or foot tap or another gesture in a camera's view or within range of a microphone.

As illustrated in an example workflow in FIG. 1, self-service subjective refraction by decision module 140, may be performed after self-service auto refraction 130. However, an auto refraction step may follow a subjective refraction step in an example process that uses the auto refraction step to check the subjective refraction test results or one or more parts of those results obtained in a prior subjective refraction test. Auto refraction may also be used to obtain an alternative visual acuity test score for the user. In addition, a self service refraction test may be performed without the use of any auto refraction before or after the subjective refraction procedure, and a self service refraction device may include both auto refraction and subjective refraction components, or just a subjective refraction component, while a separate auto refraction device may or may not be used in alternate example embodiments involving the use of a stand-alone self-service subjective refraction device or method.

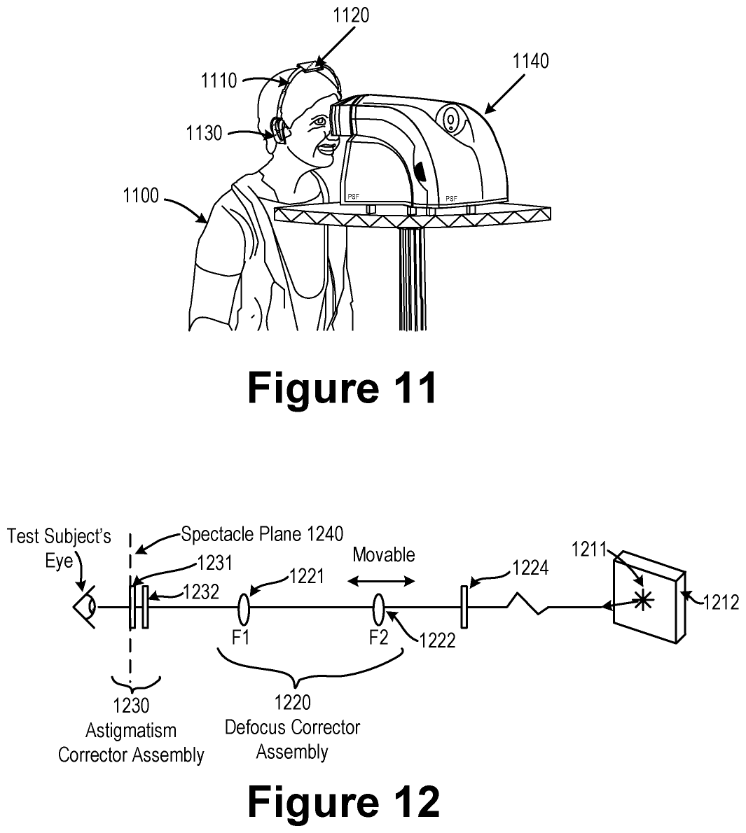

A self service subjective refraction instrument (or component of a broader use instrument) may include an optical assembly in accordance with several different configurations of lenses and/or other optical components. Examples of such an optical assembly that may be included in certain example embodiments of a self service refraction device, and examples of certain steps in a self service refraction method that may use such a device, or a different device, have been disclosed in U.S. Pat. No. 7,699,471, and in U.S. Pat. Nos. 10,383,512, 10,194,794, 8,967,801, 8,366,274, and 7,726,811, each by the same inventor Shui Lai, and each being hereby incorporated by reference. The optical assembly has adjustable optical power in sphere, cylinder and axis in certain embodiments, or just sphere and cylinder in certain embodiments, and higher order aberrations may be diagnosed and corrected in certain embodiments along with sphere and cylinder or along with sphere, cylinder and axis. The available optical powers may be aligned or positioned at the equivalent cornea plane of the user's eye during an example self service refraction exam process using a device such as one of the example embodiments illustrated in block form in FIGS. 4A-4E and/or those example embodiments that are schematically illustrated at FIGS. 11-13.

FIG. 4A illustrates a self service refraction apparatus in accordance with an example embodiment. A decision module 460 is informed by, or programmed by, or is configured in accordance with a database of standard refraction rules 470a by refraction experts or industry standards or a licensed group such as the AMA and/or a database of learned refraction rules 470b based on machine learning and computer training, e.g., using a neural network. The decision module 460 programmed by one or both databases 470a and/or 470b, with input from a test subject including choices between presented corrective optics alternatives and answers to questions provided to the test subject by the decision module 460, and in view of visual acuity test scores, is programmed to make decisions 462, 464, 466, 468 and/or 469 during a self-service refraction procedure. Among these are selecting a refraction viewing target to present to a test subject through a specific arrangement of a corrective optical assembly 462, selecting an optical component to adjust 464 by moving a lens or pair of lenses of the corrective optical assembly, selecting an optical step size increment of adjustment 466, selecting to continue a self-service refraction test or instead to end the test 468, and/or selecting questions to communicate to the test subject regarding the appearance of images of viewing targets 469, such as the shape, clarity, blurriness, brightness, size or other appearance criteria. Those decisions instruct the processor 410 to actuate a motor control unit 420 to move a lens or pair of lenses translationally or rotationally to adjust power of defocus by DCA 422, or power of astigmatism by ACA 424, or optionally axis angle of astigmatism. The decision module 460 may check visual acuity and may be informed by results of visual acuity scores at one or more stages during an example self-service refraction procedure.

FIG. 4B illustrates a self-service refraction apparatus in accordance with an example embodiment including a decision module 460 programmed by, informed by and/or configured in accordance with a rules database 470 and subjective input from a test subject to formulate decisions regarding next steps in a self-service refraction process and to communicate those decisions as commands that instruct or program an on-board processor 410 to produce on a display 450 certain viewing targets 452 and to control adjustments of sphere, cylinder and axis performed by a motion control unit 420 including motors and gears for moving optical elements such as lenses, mirrors, apertures and/or waveplates that are corrective optics components of an optical assembly that is disposed between the test subject's eye or eyes and the display 450 of the viewing target 452. In an example embodiment, corrective optics components may include an adjustable defocus corrector assembly 422 and an adjustable astigmatism corrector assembly 424 with which sphere power, cylinder power and axis of astigmatism may be adjusted in accordance with certain decisions made by the decision module 460.

The decision module 460 comprises digital code embedded in an electronic storage medium that produces output commands that instruct the processor 410 to configure a specific viewing target and a specific configuration of corrective optics for presentation to a test subject. In certain embodiments, the decision module 460 may instruct the processor 410 to communicate to the test subject, e.g., using an electronic display interface 430 or a voice interface, questions asking the test subject to compare, contrast or choose between two images of a viewing target. In example embodiments, the decision module 460 may instruct the processor 410 to communicate questions to the test subject such as, “does the viewing target appear more round” or “less elongated,” or “more red” or “more blue” or “more or less monochrome” or “does the viewing target appear more or less uniform in brightness” or “more less uniform in color” or “more or less gray,” either compared with a previous image or in an absolute sense. That is, the decision module 460 may instruct the processor 410 to communicate certain questions to the test subject regarding the viewing target's appearance, i.e., blurriness, clarity, size, shape, to the test subject either absolutely or comparatively or both. Answers to these questions are provided by the test subject by actuating an input device 440 which the processor 410 configures as digital input data to inform the decision module 460. The input device 440 may include a joystick, a keypad, a mouse, a keyboard, a touch screen, or a microphone. The decision module 460 may communicate software commands or digital instructions regarding next steps for presenting a viewing target to a test subject or eye care patient or user of a self-service refraction instrument that includes an optical assembly configured with an adjustable sphere power and with an adjustable astigmatism power, and also in an example embodiment, with an adjustable astigmatism axis angle.

The example self service refraction apparatus illustrated in FIG. 4A includes a processor 410, and a motion control unit 420 which moves optical components, as directed by the processor 410, of a defocus corrector assembly 422 or DCA 422 and an astigmatism corrector assembly 424 or ACA 424. A user interface display 430 and a patient input device 440 are configured for facilitating communication between the test subject and the self-service refraction apparatus. A viewing target display 450 is arranged such that a test subject can view any of several possible viewing targets 452 through the optical components of the DCA 422 and ACA 424. A decision module 460 provides decisions regarding ordering of steps in the refraction process, as well as selecting step size increment values for increasing or decreasing defocus power and astigmatism power, and determining when to run a visual acuity test, and when to continue or end the test based on whether a target visual acuity, e.g., 20/12 or 20/20, has been reached or whether significant further improvement in visual acuity would be likely achieved by continuing the test. A rules database 470 is shown in the example embodiment of FIG. 4A to be accessible to the decision module 460 and providing input in making decisions in accordance with industry standards based on the test data.

In one example embodiment, the optical assembly is capable of providing adjustments as follows:

1. Any one of the three primary optical parameters, elements or components of human vision known as sphere, cylinder and axis, can be changed on demand; and

2. Adjustments to each of the three components can be applied to the test subject's eye to change her vision individually by component or simultaneously with more than one component of the three components.

3. Visual Acuity (VA) tests can be performed as determined by the decision module by displaying an eye chart or certain optometric symbols of varying size and/or orientation, or a PSF target, for example, as disclosed in US patent by Shui Lai, U.S. Pat. No. 8,632,184, which is incorporated by reference, to the test subject without corrective optics to test an uncorrected visual acuity (“VA”) of the test subject, or with corrective optics interposed along an optical path between an eye or eyes of the test subject and the eye chart or other suitable viewing target to determine a corrected visual acuity using a specific prescription or potential prescription. In an example embodiment, the letters of a Snellen chart may be read out loud, and voice recognition software may be used to identify what was said, and labels or tags may be applied indicating whether the letters are correctly identified. Thereby, a computer or on-board processor of a self service refraction device in certain embodiments has the ability to automatically score the visual acuity of the test subject. Voice recognition software is commercially available from Dragon Speak, and Google Voice.

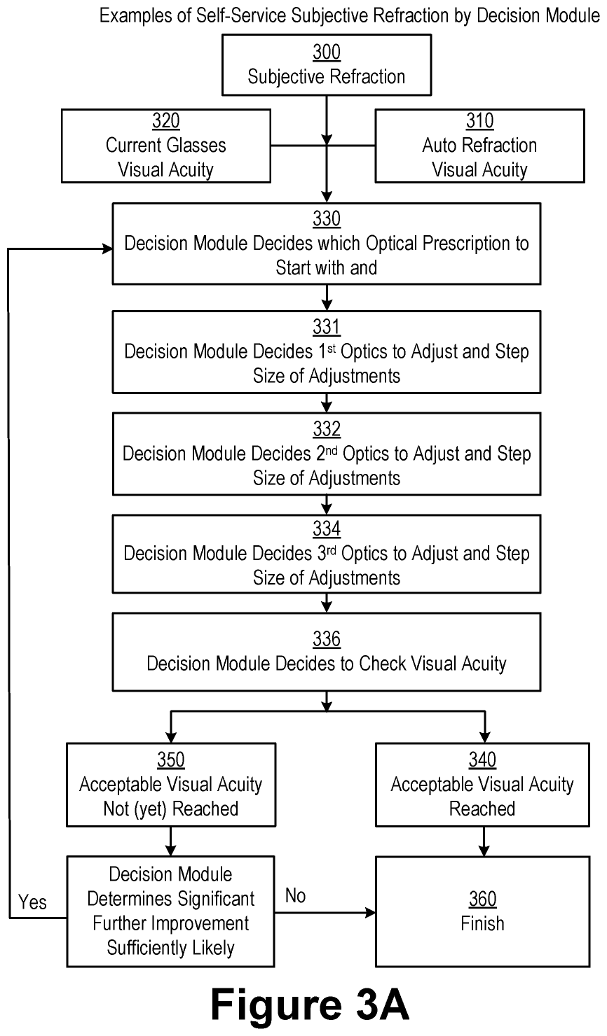

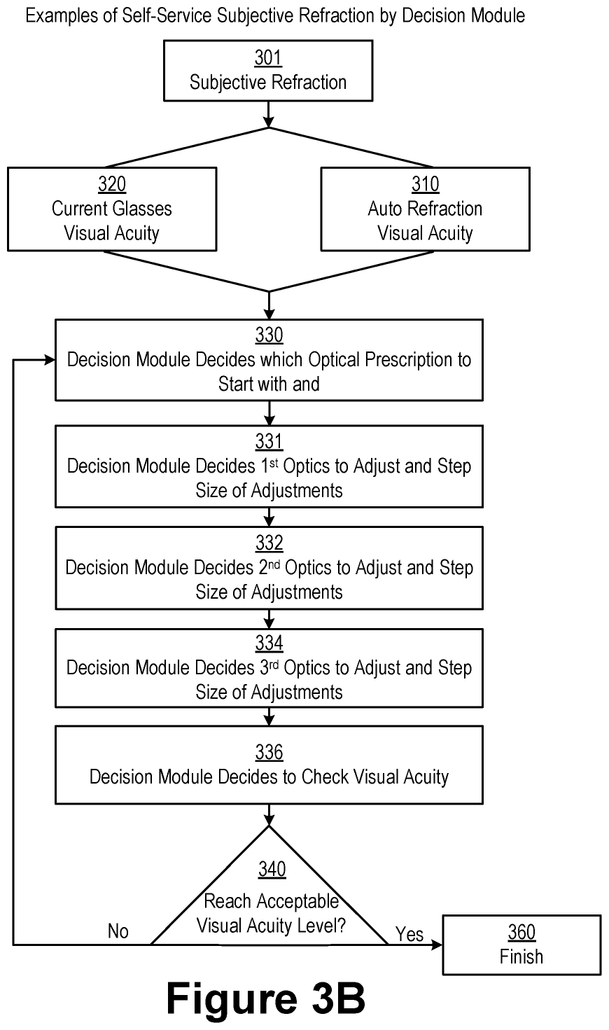

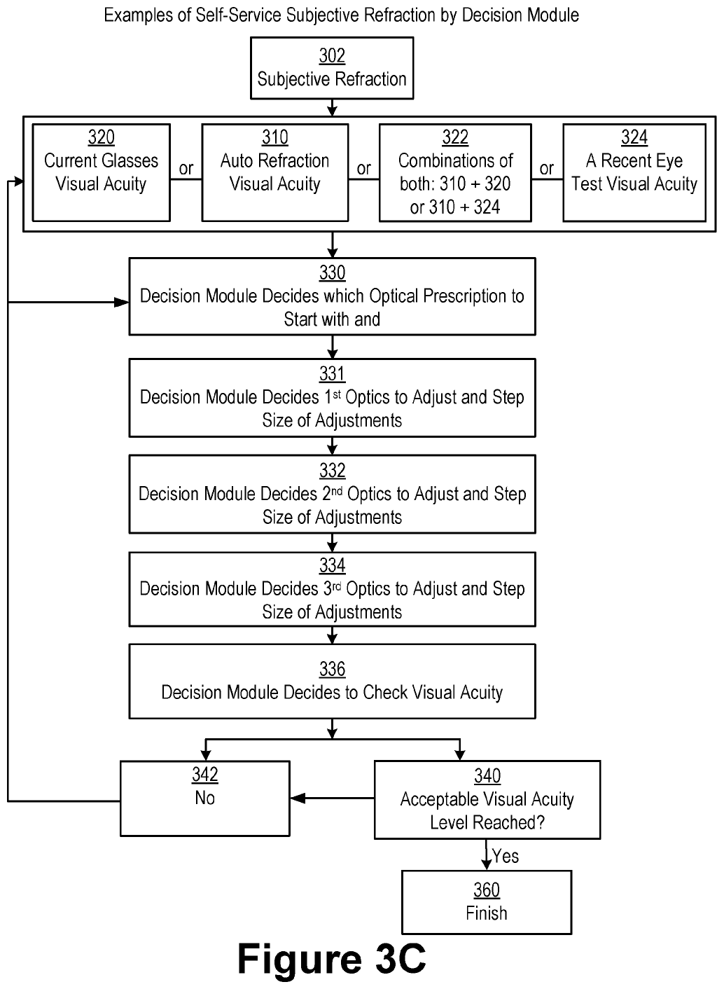

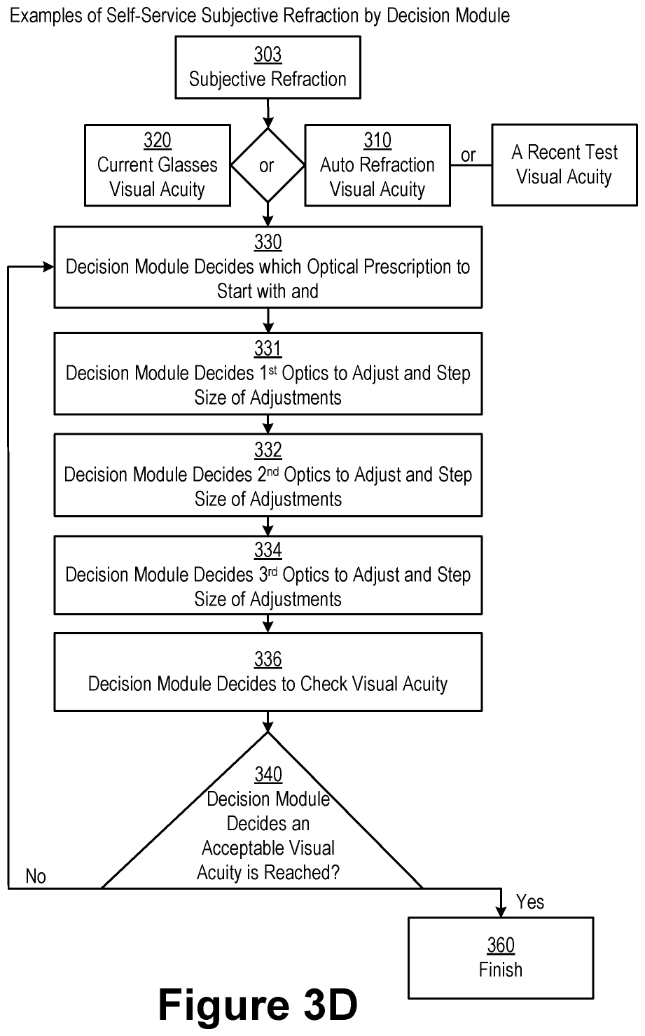

A self-service subjective refraction process in accordance with certain embodiments may start, after initialization and alignment of one or both eyes of the user along the optical path of the self service refraction device, by applying the sphere, cylinder and axis values obtained from auto refraction at 250 in FIG. 2, to test the optical assembly. In other embodiments, a current or previous prescription for the user may be applied to the optical assembly at the start of a self service refraction procedure. In the example self-service subjective refraction by decision module illustrated in FIG. 3A, a current prescription corrected visual acuity for the test subject 320 and an autorefraction test corrected visual acuity 310 are each available as input data.

The decision module 460 is programmed to select a corrective optics configuration at 330 to start the self-service subjective refraction test 300 at FIG. 3A-3D and at 140 of the example procedure of FIG. 1. In one example embodiment, the decision module 460 selects the corrective optics associated with the highest available visual acuity test score and/or a most recent visual acuity test score. In an example whereby a current prescription visual acuity 320 and an autorefraction visual acuity 310 are available, the decision module may be programmed to select the corrective optics associated with the higher visual acuity score between the two available prescriptions to start the subjective component at 330 in FIG. 3A.

Examples of a self-service subjective refraction process 140 such as the example illustrated in FIG. 1 are detailed in the self-service subjective refraction example processes 300, 301, 302 and 303 that are illustrated at FIGS. 3A-3D. Referring to FIG. 3A, the user's visual acuity is scored 310, based on the Rx from the auto refraction.

If there is a previous refraction, or the readings of the current eyeglasses' prescription 320 are available, that Rx 320 is input into the computer, by either typing it, scanning into a PDF file, a camera capture of the Rx certificate and photo image upload to the PC or other on board self service refraction device processing component or an online database of optometric prescription data may be used with the test subject's consent to upload and download prescriptions from and to, respectively, remote vision services locations and perhaps different eye examination test providers. In this example embodiment, computer software code will program the processor to read the Rx values from an image of a Rx certificate. If a test subject has an eye doctor or an optometrist that he or she has worked with in the past, then on a second or other subsequent visit either in person or virtually with this same eye care specialist, the previous visual acuity test data and prescription may be available in the test subject's personal file with his or her office.

In 320 of the example process of FIG. 3A, a visual acuity test may be then performed with the current glasses Rx 320 applied to the optical assembly, and the visual acuity may be scored and recorded. Both a current prescription visual acuity 320 and an auto refraction visual acuity 310 may be used. e.g., by performing a visual acuity check after an auto-refraction test on a user and performing another visual acuity check while wearing contact lenses or prescription glasses, for example. Then, the on board processor may be programmed for calculating a further correction to a current prescription. Alternatively, based on the visual acuity values of the auto refraction 310 and those using current contact lenses or glasses 320, the decision module 460 may calculate and make a choice between the corrective optics configurations associated with a highest visual acuity score available as input data or a combination of two close scores. That is, 310 and 320 in FIG. 3A may be exclusively alternative (either/or) options or they may be used in combinations of 310 and 320 that may be usefully applied. In one example, the decision module 460 may pick one over the other if one has a substantially higher visual acuity associated with it in the not too distant past. In another example, the decision module 460 may combine two sets of corrective prescription values by performing an averaging on the two sets of values, namely average of the sphere, cylinder power and axis, component by component. In another example, multiple past prescriptions of the test subject may be available and determined by the decision module 460 as trending in a certain direction over time, such that the decision module 460 may extrapolate from the trend data of multiple previous prescriptions based on those previous dates and the current date to use an expected or extrapolated current prescription as a starting point for a preliminary visual acuity test and subsequent self-service refraction eye exam in accordance with example embodiments.

### Baseline Visual Acuity

The test subject's visual acuity will be measured in one embodiment using a traditional Snellen chart. The decision module 460 uses the visual acuity based on the prescription 320 of the current eyeglasses or other corrective lenses, e.g., contact lenses or intraocular lenses, and the visual acuity of the auto refraction prescription 310 outcome from the wave front measurement. This forms a basis of the test subject's vision potential in the example embodiments of FIGS. 3A-3B. The decision module 460 takes into account a visual acuity from a recent eye test at 324 in the example embodiments of FIGS. 3C-3D. In the example embodiment of FIG. 3C, the decision module 460 takes into account combinations of visual acuities at 322 based on autorefraction 310, a current prescription 320 and a recent eye exam 324 in determining a starting point for a self-service subjective refraction eye test in accordance with example embodiments.

In one embodiment, the computer is configured to recognize the test subject's voice, with a built in voice recognition software, wherein the computer may score the visual acuity test from the test subject's voice response. In another embodiment, the test subject's face may be recognized or the test subject's file may be accessed upon the user logging in to the self-service refraction kiosk or upon the user inserting a card and entering a pin.

### Decision Module

A decision module 460, as illustrated in example embodiments at FIGS. 4A-4E, may be programmed in accordance with one or more databases containing refraction procedure rules from physicians and refraction technicians and other experts and/or by empirical machine learning processes and/or Bayesian statistical techniques. A set of refraction rules 470 may be utilized and updated with input from one or more experienced refraction experts, including ophthalmologists, optometrists, eye care technicians, and/or statistical results of large numbers of refraction eye examination procedures and/or if available, from the previous history of visual acuity tests and prescribed corrective vision profiles of the same self-service subjective refraction test subject whose eyes are being self-tested once again perhaps after the passage of time and/or upon experiencing regular or unusual eye trauma. By observing and recording how an experienced refractionist would decide what to do next on various refraction scenarios, a decision module 460 in accordance with example embodiments is trained and may continue to learn to perform similarly with similar situations. The decision module 460 may also be further trained by machine learning processes, including training by association of visual acuity improvements with changes in optical component values.

In one subset of example embodiments, the decision module 460 may include and/or may be programmed in accordance with:

(a) a set of industry established refraction rules 470 provided by one or more refraction experts, wherein these rules guide the refraction process, to attend improvement in visual acuity, and in a short processing time. In an example embodiment, the sphere component is first adjusted to reach its maximum visual acuity, followed by adjustment of the cylinder power component to reach its maximum visual acuity. Then the axis component is adjusted to reach its maximum visual acuity. Based on the progression of the improvement of the visual acuity in the process, the decision module 460 may decide whether to continue to refine the individual components. The decision module 460 may determine that a continuing improvement is sufficiently likely and would be sufficiently significant, such that the user may have not reached her maximum visual acuity potential yet, and continue the self-service subjective refraction test process. Alternatively, the decision module 460 may decide to stop any further adjustment if the visual acuity level has reached a high level visual acuity, such as at 20/12 or better, or another visual acuity target such as 20/20. Alternatively even if the visual acuity is not at 20/12 or better, but the decision module 460 could conclude that the user's maximum visual acuity potential had been reached, e.g., based on comparing and contrasting data received in a current test with a library of stored reference data sets and/or by inputting current test data to one or more accepted formulae or consulting a look-up table stored within the decision module 460 and/or in an accessible database, and further adjustments would not be likely to produce meaningful improvement in the visual acuity for this user, then the decision module 460 may decide to end the test; and/or

(b) a set of learned refraction rules 470 compiled with a learning algorithm and/or Bayesian software, e.g. that may be based on a neural network trained to make decisions based on one or more of the following factors: (i) a visual acuity score at the present or current optics setting, (ii) an extent of improvement in visual acuity score between current and previous optics settings, (iii) a priority rule for selecting which of a selected subset of optic components, e.g., the three optic components of sphere, cylinder and axis, is to be tested next, based on an extent of visual acuity improvement in each of the three optical components, or alternatively based on just one or two of the sphere, cylinder and axis optical components and/or one or more additional, perhaps subtler, optical components. A decision module 460 in alternative embodiments may include (a) or (b), including any combination of (i), (ii) and (iii), or a combination of both (a) and (b).

After a decision module 460 in accordance with any of these example embodiments is sufficiently trained or otherwise equipped with a sufficient database of industry-established, expert-provided and/or learned rules, to base logical, consistent decision-making during self-administered eye tests at efficient self-service refraction kiosks, like efficient self-service banking at ATMs, the decision module 460 may be configured to make certain decisions mandated as certain logical rules-based conclusions drawn from applying the rules database 470 available to the decision module 460 to the test data which indicates the validity of those certain conclusions as being in accordance with those rules as applied to that test data. Decisions that the decision module 460 may be configured to make may include:

i. Which of the three optical components should be adjusted next? In an example, many optometrists would start with adjusting the sphere component, then the cylinder power, then the axis angle. The decision module 460 may determine to re-test one or more of the sphere, cylinder and/or axis components, particularly following testing and improvement of another component by adjusting an optical parameter of the test subject's corrected vision. The decision module 460 may determine not to test an optical component that has been determined to be optimized already or to be unlikely to improve by testing it now. The order of the testing may be different, particularly for test subject's known to have predominant cylinder or axis issues or issues with higher order aberrations;

ii. How big the change in the step size of an optical component is appropriate? Based on traditional optometric practice, the decision module 460 may determine to start a test using a starting step size of 0.25 Diopters, or 0.375 or 0.5 diopters or 0.125 or 0.0625 diopters. The decision module 460 may determine to reduce the step size after a coarse adjustment portion of a test during a refinement portion of the test to achieve better visual acuity. The decision module 460 may use a larger or smaller starting step size based, e.g., on time since a test subject's last test, or on an extent of total correction or past prescription history of the test subject, or the test subject's age or occupation or other health issues or personal or family health history, or based on test subject-specific information that suggests a different starting step size should probably be used. While generally, the step size may be mostly or only decreased throughout most tests, step size may be increased one or more times during some tests in some embodiments. The step size may be increased to a slightly larger step size than a just previous step size in most circumstances when a step size increase is determined by the decision module 460. The decision module 460 would rarely determine to increase a step size to larger than the starting step size, and would not likely do so during a refinement phase of a test, while a typical coarse adjustment phase of a test would rarely include an increase of step size to greater than the starting step size, unless the decision module 460 determines early in the test that the starting step size should have been greater than the starting step size was for this test. A test subject's pattern of choices may suggest that one or more of those choices has caused a divergence away from an optimal end point for an optical component that is being tested, such that the decision module 460 may correct for the divergence with a slightly larger step in the opposite direction. A next step size may be reduced to half or a third or a quarter or less of a current step size when the test subject indicates that a present image appears to be almost perfectly clear or optimally focused. A reduced step size may be selected based on an absolute or percentage or fractional amount of change of step size from the previous step size or on an estimated distance from an optimal end point based on past experience.

iii. When does an optical component reach its optimal point, such that no further meaningful improvement should be attempted? If the decision module 460 determines that a test subject's visual acuity has already reached 20/12 or 20/10 or another preset threshold or target vision quality, then the test may be stopped notwithstanding that no other indicators suggest that the test subject's optimal vision quality has likely been established. If the decision module 460 determines that the test subject has provided contradicting answers when prompted by the decision module 460 to make a choice between same or similar pairs of viewing targets, such that each of the contradicting answers was likely based on small perceived differences in focus and/or clarity of one viewing target over another, or was even a perceived coin flip for the test subject due to the patient's sensitivity limit having been reached or due to the optical end point being nearly half way between the two choices presented to the test subject.

iv. When to check visual acuity score before, during or after a test to determine whether or not a visual acuity score has changed a preset meaningful amount indicating that the test subject' quality of vision has been improved a sufficiently significant amount?

v. When to repeat steps (i) to (iv) until the visual acuity score has not changed by a preset meaningful amount, for example; the preset amount can be at less than two lines of letters of improvement in a visual acuity test, or it could be set at less than one line of letters of improvement. The decision module 460 can be programmed in certain embodiments to determine when the visual acuity reaches the definition of no meaningful improvement, and therefore the refraction test is finished.

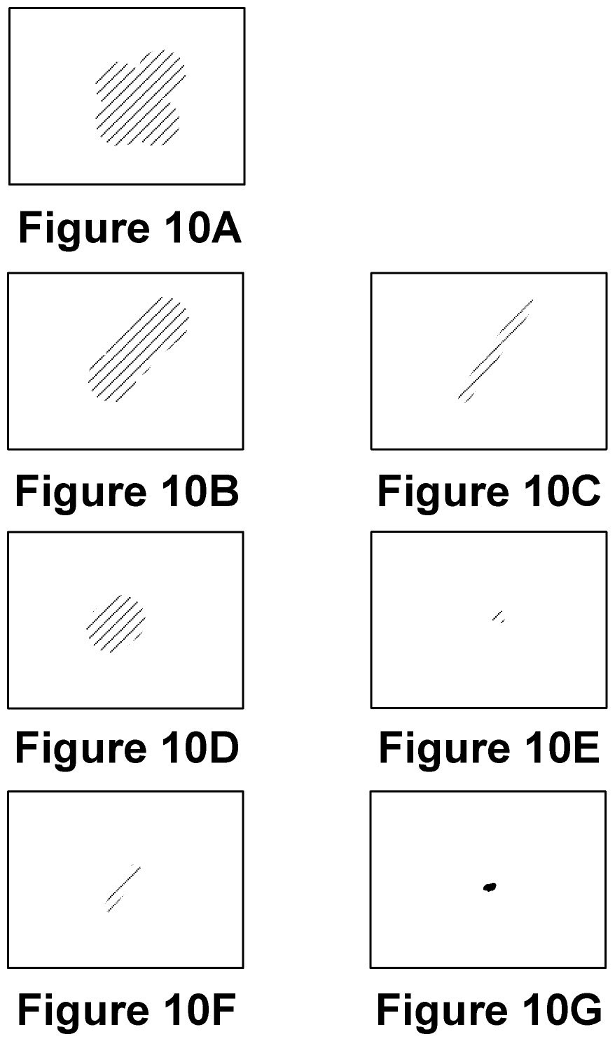

Next, in an example embodiment, collected data including one or more of an Rx from autorefraction 310, a current glasses Rx 320, and their respective visual acuity scores may be input into the decision module 460 at step 330 in the example self-service refraction processes illustrated at FIGS. 3A-3D. Based on these input visual acuity scores, the decision module 460 will determine at 330 an optical prescription at which to begin a self-service subjective refraction eye test in accordance with an example embodiment. Based on the training and/or other accessible refraction rules 470, the decision module 460 will decide in certain embodiments which one, either the sphere, cylinder, or the axis is to be adjusted first as shown in 331 in the four example embodiments illustrated at FIGS. 3A-3D. The optics assembly is then automatically adjusted by moving an optical component a certain amount or replacing one lens with another, for example, to adjust the optical parameter (e.g., sphere, cylinder or axis) determined by the decision module 460 to be adjusted first at 331, and by a step size increment (e.g., some fraction of a diopter or of a tenth of a diopter) and in a direction (e.g., increasing or decreasing) that are also determined by the decision module 460 (e.g., ±0.0675 diopters, or ±0.125 diopters or ±0.25 diopters or ±0.375 diopters or ±0.5 diopters). Then, the decision module 460 decides which optical parameter (sphere, cylinder, or axis) will be the second to be adjusted at 332 of FIGS. 3A-3D, and which will be the third to be adjusted at 334 of FIGS. 3A-3D, and when to check visual acuity at 336. The decision module 460 may then decide at 340, based on the results of the visual acuity test performed at 336 whether to end testing at 360, or instead continue testing by returning to step 330 or 331 and retesting one or two or all three of the sphere, cylinder and axis components one or more additional times and in which order. For example, by choosing to test in an alternative, viable order the sphere, cylinder and axis components, the decision module 460 may have determined that a probability exists that improved visual acuity may be a result. Examples of different orders of testing the sphere, cylinder and axis are described with reference to FIGS. 10A-10G below. The decision module 460 may decide which of the sphere, cylinder and axis, if any, should not be tested either initially or when other optical parameters are being retested, and which, if any, other higher order optical parameters should also be included in a self-service subjective refraction eye test.

Several example embodiments of refraction eye tests involving objective wavefront refraction using wavefront aberrometry devices and methods and/or certain subjective refraction steps involving input from the test subject have been described in detail in U.S. Pat. Nos. 7,699,471, 10,383,512, 9,320,426, 7,726,811 and 8,366,274 and other above-referenced patents by the present inventor Shui T Lai, all of which are incorporated by reference. For example, details as to how certain adjustments may be performed in certain example embodiments in accordance with steps 330-334 of FIGS. 3A-3D are described in several example embodiments in the '471 patent and in the '512 patent, and for each of the sphere, cylinder and axis optical components, as well as testing and correcting for certain higher order optical aberrations. Based on the visual acuity test score obtained at 336, the decision module may determine that an acceptable visual acuity has been reached at 340 in FIG. 3A and end the test at 360. The decision module 460 may alternatively determine that an acceptable visual acuity has not been reached at 350 of FIGS. 3A and 3C. If the decision module 460 determines that another iteration of testing may produce an improvement of visual acuity, then the decision module 460 may decide to continue the process by returning to 330 of the test, or if the last optical arrangement is defaulted as the starting prescription for the continued testing, then the process may return directly to step 331 of the test. If the decision module 460 determines that another iteration would not likely produce a significant improvement of visual acuity, then the process may end despite a target visual acuity not being reached.

Another example method for adjusting a sphere component in a self-service subjective refraction eye test involving a decision module 460 that takes into account objective and subjective input data in making decisions automatically based on expert learning and/or reference to a refraction rules database as part of a digital device involving sophisticated automatic processing will now be described as a next example. In certain embodiments, two presentations of a viewing target manifest two different sphere values of corrective optics through which the test subject is disposed to observe the viewing target. The difference between the two sphere values, or step size, presented to the test subject is determined by the decision module 460 based on a general strategy of gradually decreasing the differences in sphere values of the choices presented to the test subject and on a demonstrated sensitivity of the test subject to sphere value adjustments between sets of two or more viewing targets. The test subject may be asked by a voice prompt or words or other symbols on a display screen or noise prompts or vibrations, for example, to decide which one of the two or more choices is more clear, or more focused and/or which of the choices is least clear or least focused.

The test subject, who is using the self-service refraction device to help determine his or her own corrective optical prescription profile may respond by communicating a choice by voice or actuation of an input device or by typing an answer. For example, the test subject may communicate that: “choice 1 is better than 2.” Or vice versa. If choice 1 corresponds to an optical arrangement that provides a higher sphere value than that providing choice 2, then the test subject is understood to have communicated that she prefers the higher sphere value option among the previous choices, and the decision module 460 may as a result in certain embodiments move the sphere setting towards higher values at a certain step size of sphere power increments based on that subjective input from the test subject. The decision module 460 may next present two additional choices of viewing targets to the test subject corresponding to observing a viewing target through choices of optics that have different sphere powers and are configured on average to have higher sphere powers than the average of the previous choices presented to the test subject.

The process may iteratively continue, with prior choices communicated by the test subject informing the decision module 460 in determining next sphere power adjustments, in step size, in changes in step size, and/or in direction of incremental step size differences in sphere power between pairs or larger groups of three or more presented viewing targets, until in certain example embodiments the decision module 460 determines that either:
1. The test subject has reversed the direction, from moving towards higher sphere values to preferring a lower value, or vice-versa. The decision module 460 next determines whether this reversal is indicative of the step size being too large, such that the optimal end point may have been passed over and lies between the last two selected sphere values. The decision module 460 may determine to reduce the step size and continue to move the sphere in this example either in the reverse direction starting from the last sphere value presented or in the same direction starting from the next to last sphere value presented, and in either case using a smaller step size than the one that was determined to have been too large. Thereby the decision module 460 approaches an optimal end point getting closer and closer in smaller absolute increments while in percentage terms the increments may be constant or even increasing, until a sensitivity limit is reached in this refining process; or
2. The test subject indicates that there is no apparent or discernible difference between the two choices presented.

The decision module 460 may decide to stop any further investigation in sphere in this example, and instead may decide to move on to investigate cylinder or axis. The decision module 460 may be configured to change the size of the step based in part on test subject input to assist in getting to the correct, precise, accurate, improved, enhanced and/or optimized end point more efficiently in certain embodiments.

### Astigmatism Correction

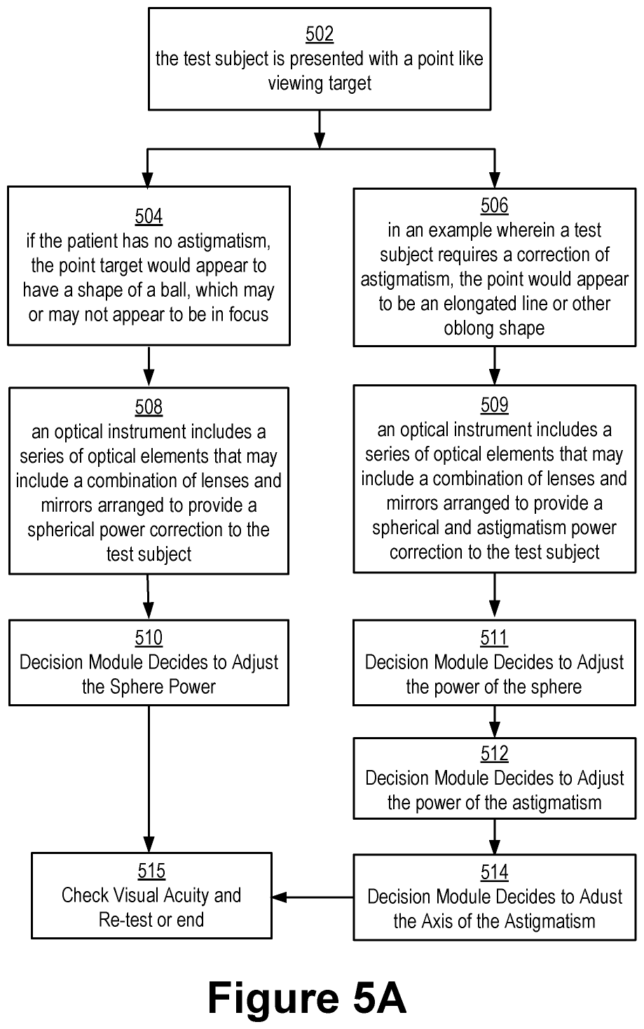

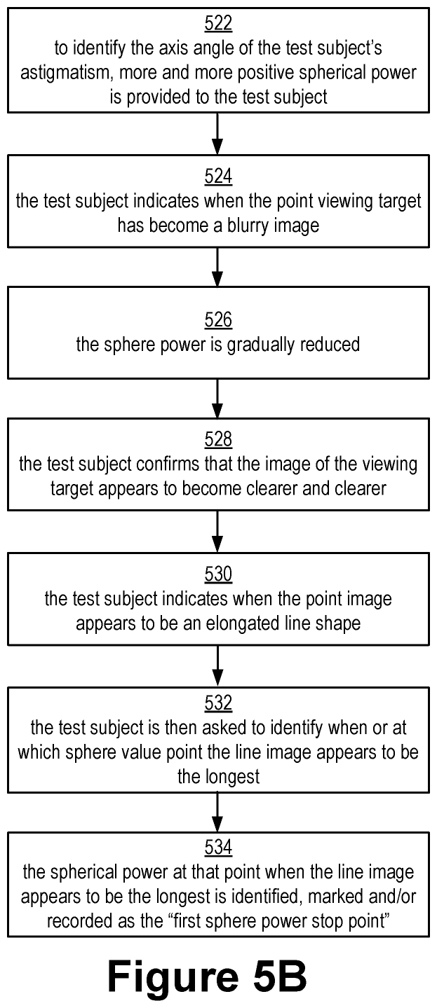

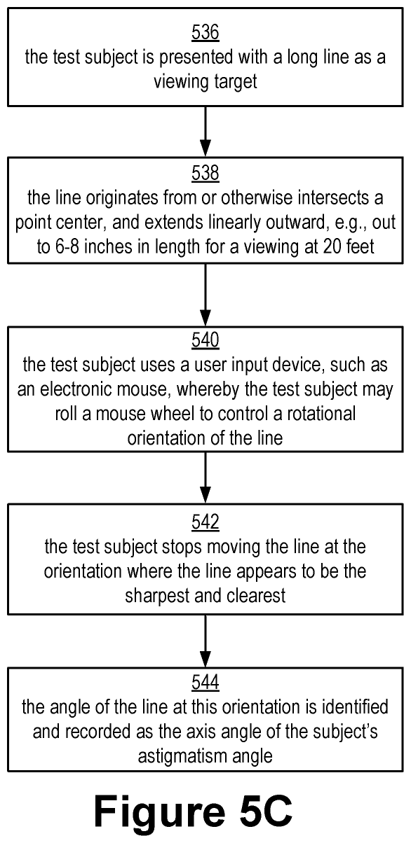

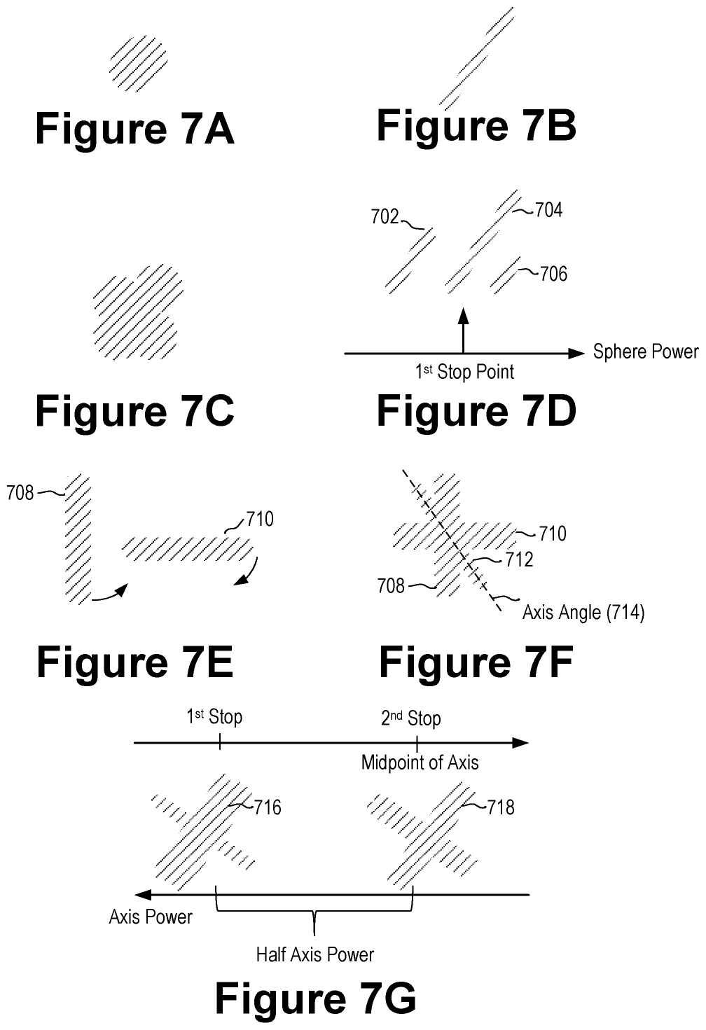

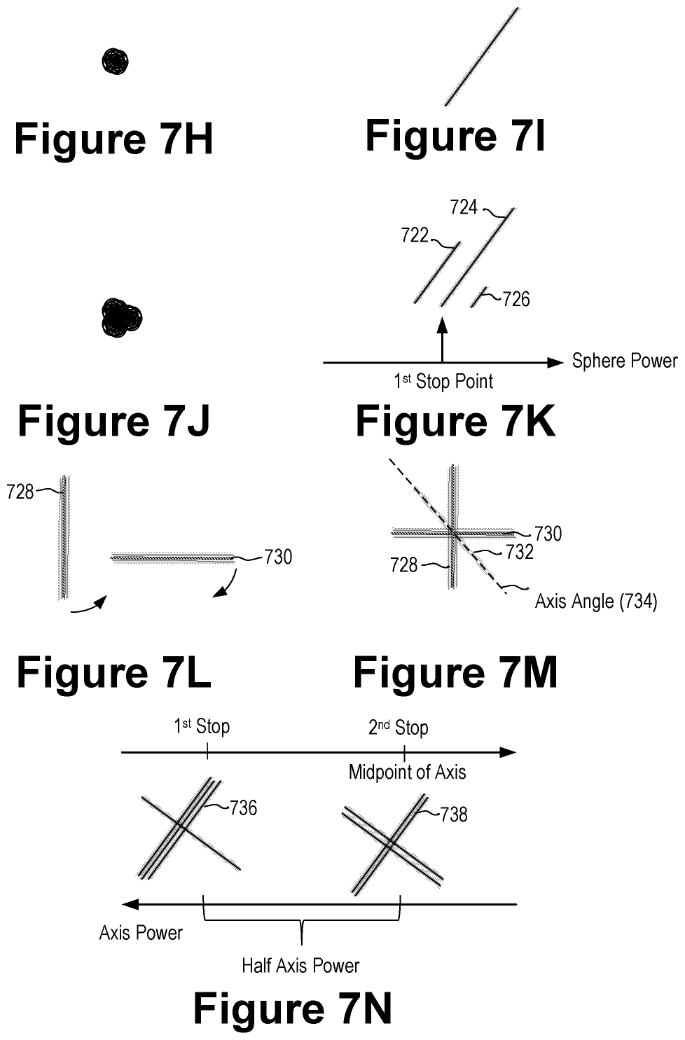

A decision module 460 in accordance with certain embodiments may be configured to determine an astigmatism correction for a test subject in accordance with any of the following example embodiments, referring specifically now to FIGS. 5A-5C, 6 and 7A-7G. To correct astigmatism in a self-service refraction test, the decision module may be configured to correct both the axis angle and the power of the astigmatism. In certain embodiments, the decision module determines to present a test subject with a point like viewing target 502. If the patient has no astigmatism, the point target would appear to have a shape of a ball, which may or may not appear to be in focus 504, as in the example of FIG. 7A. However, in an example wherein a test subject requires a correction of astigmatism, the point would appear to be an elongated line 506, as in the example of FIG. 7B.

The decision module 460 may configure an optics assembly of a self-service refraction device instrument through which the test subject will look at one or more viewing targets, which may be point-like viewing targets as in 502 of FIG. 5A. The optics assembly may include a series of optical elements that may include a combination of lenses and mirrors. FIG. 5A is a split flow chart illustrating two examples of self-service refraction by decision module 460 processes in accordance with example embodiments, including a first process of the decision module 460 selecting a first subset of optics 508 to test when the test subject has an astigmatism issue 506, as understood from the test subject's input indication that the point-like viewing target 502 appears to be an elongated line or other oblong shape 506, as well as sphere and axis issues, and a second process of the decision module 460 efficiently selecting a second, smaller subset of optics 509 to test when the test subject has a sphere issue but does not have an astigmatism issue 504, as understood from test subject indication that a point like viewing target 502 appears to have the shape of a ball or other circularly symmetric object 504.

The decision module 460 may arrange the optics assembly in a way that provides a selected spherical and astigmatism power correction to the test subject 509, based on previous and/or current test data, and on said expert refraction rules database and/or on learned or trained refraction rules, and wherein the power of the sphere 511 and power of the astigmatism 512 are both adjustable in certain example embodiments. The axis of the astigmatism 514 may also be adjustable in certain example embodiments. In the case where the test subject does not have an astigmatism issue, the decision module 460 may decide only to adjust the sphere power 510. In either case, the decision module 460 will check visual acuity 515 in both of the example embodiments illustrated at FIG. 5A, and will decide whether to retest one or more components or end the test 515 based on results of the visual acuity test. Examples of existing optical instruments that may be used or modified for use are described in U.S. Pat. Nos. 10,383,512, 9,320,426, 8,967,801, 8,950,865, 8,790,104, 8,632,184, 8,632,183, 8,388,137, 8,366,274, 8,033,664, 7,954,950, 7,726,811, 7,699,471, 7,695,134, 7,461,938, and 7,293,871, which are all incorporated by reference.

FIG. 5B illustrates a first part of an example method wherein the decision module 460 decides to identify an axis angle of a test subject's astigmatism. A point viewing target is provided for the patient to observe as a point as illustrated at FIG. 7A. More and more positive spherical power is provided to the test subject at 522, until the test subject sees at step 524 a blurry image of the point viewing target such as that illustrated at FIG. 7C. Now the sphere power is gradually reduced at 526. The test subject would confirm that the image of the viewing target appears to become clearer, and clearer 528, until the test subject indicates at 530 that the blurry image appears to be an elongated line shape as illustrated at FIG. 7B. The test subject is then asked at 532 to identify when or at which sphere value point the line image appears to be the longest. The test subject would see the shorter lines 702 and 706 on either side of the longer line 704. The spherical power at that point where the test subject sees the line 704 is marked at 534 as the first sphere power stop point as illustrated at FIG. 7D.

FIG. 5C illustrates a second part of an example method for identifying an axis angle of a test subject's astigmatism. After the first sphere power stop point is identified and recorded as illustrated at FIG. 7D, the test subject is presented at 536 with a long line as a viewing target such as that illustrated at FIG. 7B. FIG. 7E illustrates a line that originates from a point center, and may in an example embodiment extend out to about 6-8 inches in length for a viewing at 20 feet as in step 538. FIG. 7E shows the line rotated to a vertical line 708 and rotated 90 degrees counterclockwise to become a horizontal line 710. Using an electronic mouse, joystick, VR input, voice input, or other user input device, the test subject may, for example, roll a mouse wheel to control a rotational orientation of the long line at step 540, which originates from the previous point image center. The subject is asked to stop moving the line at step 542 when the line appears to be the sharpest and clearest line 712 as illustrated in FIG. 7F which is the orientational axis angle 714 of the test subject's astigmatism. In an alternative embodiment, the decision module 460 moves an optical component automatically which causes rotation of the line in clockwise or counterclockwise direction, until the test subject indicates that the sharpest and clearest orientation of the line has been passed. The rotation direction in this alternative embodiment is automatically reversed and the test subject stops the movement with a voice command or by actuating an input device. This angle of the line 712 at which the test subject sees the line most clearly and sharply is identified and recorded at 544 as the axis angle 714, as illustrated at FIG. 7F, of the test subject's astigmatism.

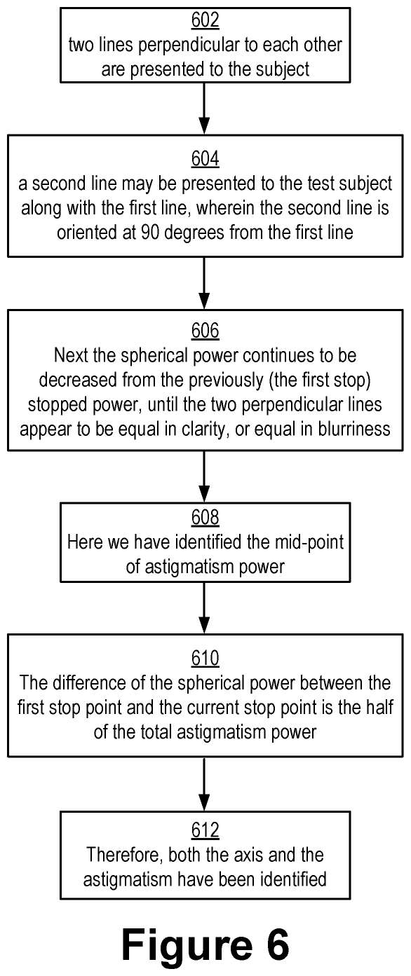

FIG. 6 illustrates an example method for determining an astigmatism power of a test subject. Now two lines perpendicular to each other are presented at step 602 to the test subject, e.g., as illustrated by the viewing target 716 at FIG. 7G. For example, in addition to the first line as just presented in the second part of the method for identifying an axis angle as illustrated at FIG. 7F, a second line of equal or similar length and thickness is presented at step 604 at approximately 90 degrees from the first line as illustrated at FIG. 7G. Note that while the perpendicular lines may be identical as presented on the display 450, a test subject needing correction of an astigmatism issue may see one of the lines more clearly than the other, as in the example of how the test subject sees the viewing target 716 in FIG. 7G.

In certain example embodiments, the two elongated shapes that are overlapped in perpendicular disposition may form a cruciform shape in FIG. 7G. The two elongated shapes in FIG. 7G may form a cross by intersecting the first elongated shape of FIG. 7G at its center with the second elongated shape. The first elongated shape may be horizontal relative to the test subject's orientation, while the second line may be vertical, in one example, while both elongated lines may be diagonally disposed and overlapped to form an “X” shape such as the viewing target 716, or a T shape, an E shape, a L shape, an H shape, or another letter shape that may be used that includes at least one pair of perpendicularly intersecting line segments such as the two cross shaped objects illustrated side by side in FIG. 7G. The second line may be, e.g., of equal length and thickness as the first line or of another known absolute or relative length and thickness, and may be presented to the test subject along with the first line, wherein the second line is oriented at 90 degrees from the first line.

In an example embodiment, the two perpendicular lines may form a cross by extending the length of one the lines through the center of the other line, or one line may bisect the other line. The two lines may alternatively form a “T” or an “L” or the two lines may extend radially outward from a single point, or may extend radially outward from a circle of finite radius, wherein the two lines may in certain embodiments while not in others, be extrapolated into the circle as intersecting at its center. In other embodiments one or both lines are disposed tangent to the circle, and either line may be perpendicular or tangent to third arbitrarily open or closed shape having continuous curvature throughout, which may or may not change along one or more surfaces and which may have one or more discontinuities in form or curvature along a closed path or from one end to another along an open path. Further examples are provided at U.S. Pat. Nos. 10,194,796 and 10,194,794, which are incorporated by reference.

Next, the decision module 460 may continue to reduce the spherical power from the previously identified and recorded first sphere power stop point illustrated at FIG. 7D, until the two perpendicular lines appear at step 606 to the test subject to be equal in clarity, or equal in blurriness, as in the X shaped viewing target 718 on the right in FIG. 7G. Here at step 608, the decision module 460 has identified the mid-point of astigmatism power as illustrated by the cross shaped object 718 on the right in FIG. 7G. The difference of the spherical power between the first sphere power stop point identified at 514 in FIG. 5A and illustrated at FIG. 7D, and in the example starting viewing target 716 of FIG. 7G, and the second stop point, illustrated as the transformation of viewing target 716 into viewing target 718 at FIG. 7G, is the half of the total astigmatism power which is recorded by the decision module 460 at step 610. Therefore, both the axis angle of the astigmatism, and the astigmatism power, have been identified for the test subject as indicated at step 612.

FIGS. 7H-7N illustrate another self-service subjective refraction eye test for a different test subject. A point viewing target is provided for the test subject to observe as a point as illustrated at FIG. 7H. More and more positive spherical power is provided to the test subject until the test subject sees a blurry image of the point viewing target such as that illustrated at FIG. 7J. Now the sphere power is gradually reduced, and the decision module 460 would ask the test subject to confirm that the image of the viewing target appears to be getting clearer, and clearer. The decision module 460 would also ask the test subject in an example embodiment to indicate that the blurry image appears to be an elongated line shape as illustrated at FIG. 7I. The decision module 460 then asks the test subject in an example embodiment to identify when or at which sphere value point the line image appears to be the longest. The test subject would see the shorter lines 722 and 726 on either side of the longer line 724. The spherical power at that point where the test subject sees the line 724 is marked by the decision module 460 as the first sphere power stop point as illustrated at FIG. 7K.

After the first sphere power stop point is identified and recorded as illustrated at FIG. 7K, the decision module 460 decides to present the test subject with a long line as a viewing target such as that illustrated at FIG. 7I. FIG. 7L illustrates a line that originates from a point center, and may in an example embodiment extend out to about 6-8 inches in length for a viewing at 20 feet as in step 538. FIG. 7L shows the line of FIG. 7I rotated to a vertical line 728 and rotated 90 degrees counter-clockwise to become a horizontal line 730. Using an electronic mouse, joystick, VR input, voice input, or other user input device, the test subject may, for example, roll a mouse wheel to control a rotational orientation of the long line, which originates from the previous point image center. The decision module asks the subject to stop moving the line when the line appears to be the sharpest and clearest line 732 as illustrated in FIG. 7M. In an alternative embodiment, the decision module 460 moves an optical component automatically which causes rotation of the line in clockwise or counter-clockwise direction, until the test subject indicates that the sharpest and clearest orientation of the line has been passed. The rotation direction in this alternative embodiment is automatically reversed and the test subject stops the movement with a voice command or by actuating an input device. This angle of the line 732 at which the test subject sees the line most clearly and sharply is identified and recorded by the decision module 460 as the axis angle 734, as illustrated at FIG. 7M, of the test subject's astigmatism.

Now two lines perpendicular to each other are presented to the test subject, who sees the “X” shaped image 736 as crossed lines of different clarity due to the test subject's astigmatism, e.g., as illustrated by the viewing target 736 at FIG. 7N. For example, in addition to the first line as just presented in the second part of the method for identifying an axis angle 712 as illustrated at FIG. 7M, a second line of equal or similar length and thickness is presented at approximately 90 degrees from the first line as illustrated at FIG. 7N. Note that while the perpendicular lines may be identical as presented on the display 450, a test subject needing correction of an astigmatism issue may see one of the lines more clearly than the other, as in the example of how the test subject sees the viewing target 736 in FIG. 7N.

Next, the decision module 460 may continue to reduce the spherical power from the previously identified and recorded first sphere power stop point illustrated at FIG. 7K, until the two perpendicular lines appear to the test subject to be equal in clarity, or equal in blurriness, as in the X shaped viewing target 738 on the right in FIG. 7N. Here, the decision module 460 has identified the mid-point of astigmatism power as illustrated by the cross shaped object 738 on the right in FIG. 7N. The difference of the spherical power between the first sphere power stop point identified as illustrated at FIG. 7K, and in the example starting viewing target 736 of FIG. 7N, and the second stop point, illustrated as the transformation of viewing target 736 into viewing target 738 at FIG. 7N, is the half of the total astigmatism power which is recorded by the decision module 460. Therefore, both the axis angle of the astigmatism, and the astigmatism power, have been identified for another test subject.

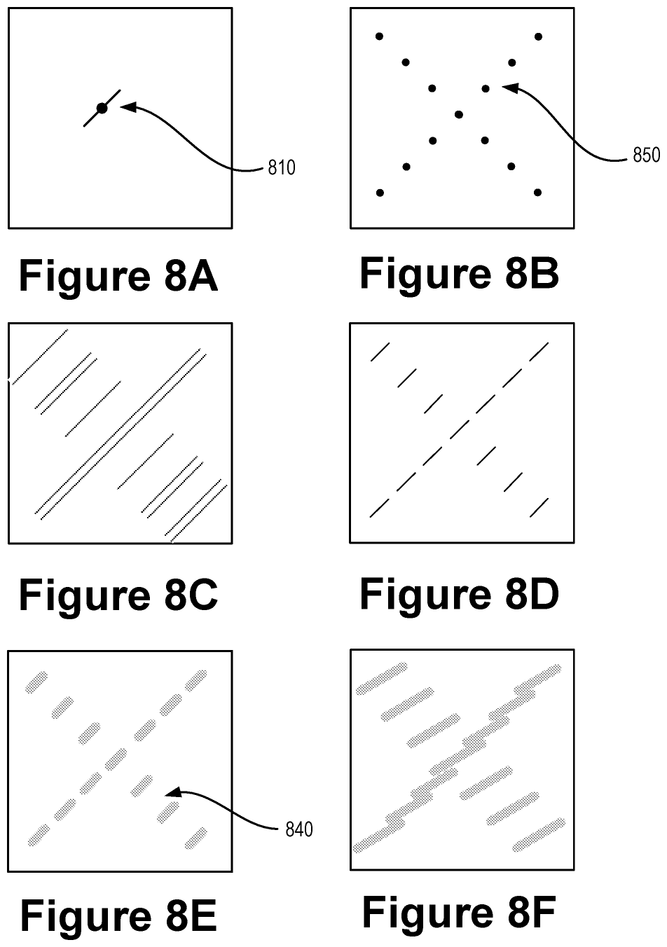

Referring now to FIGS. 8A-8E and 9 and 10A-10G, example embodiments of a method and apparatus for self-service refraction with decision module are described as configured for finding a corrective lens prescription to enhance the visual acuity of an unassisted user of the apparatus and method. Methods and self-service refraction instruments in accordance with example embodiments advantageously achieve in stepwise manner reaching a number of end points leading to enhanced refraction correction within a predetermined accuracy level. The sphere, cylinder and axis values generated by the device and method may be used in a prescription for eyeglasses or other corrective lenses such as contact lenses or intraocular lenses. This subjective refraction correction may also be used to construct a corneal tissue ablation profile and to apply it in refractive laser surgery.

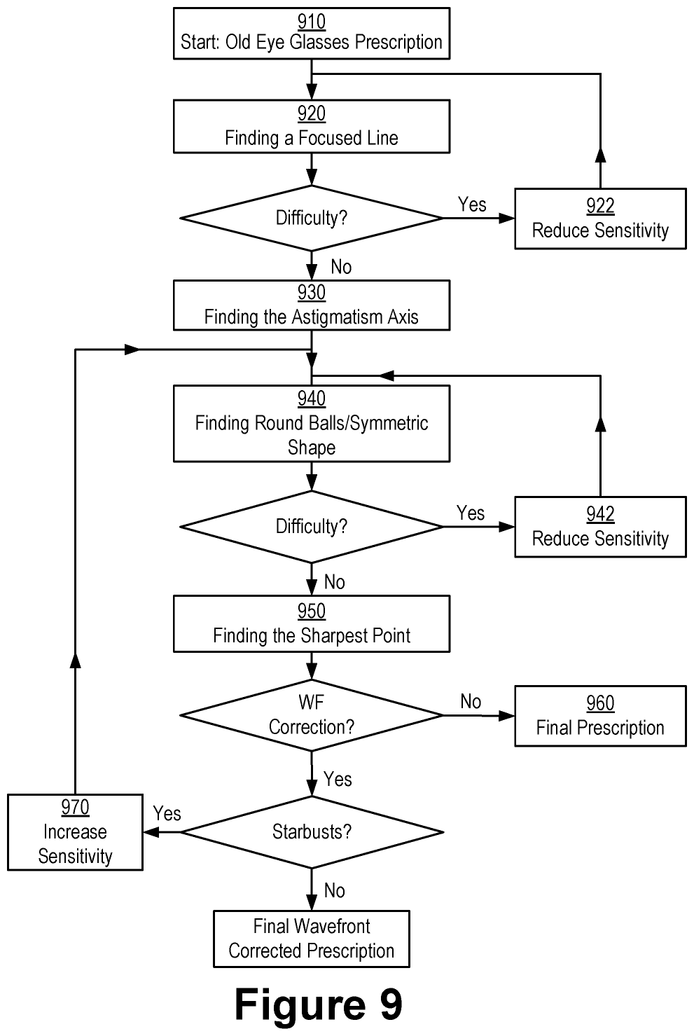

The automated refraction process includes finding specific end points. Some details have been provided above and thus are incorporated and not otherwise repeated here. Referring now to FIG. 9, at step 910, the decision module 460 may decide to use an old prescription as a starting point for a self-service refraction test. Using old eyeglasses or autorefractor values, or an arbitrary or average location, e.g., a 20/20 location, are alternative options to use as the starting point at 910 of FIG. 9, the decision module 460 of FIG. 4 instructs the processor 410 or CPU 410 to set a start and end position for the defocus corrector assembly 422 or DCA 422. For example, the old eyeglasses prescription may be −2.75D, −1.50D at 120 degrees (D stands for diopters), and note that a negative cylinder convention is used in this example. The decision module 460 may in an example embodiment first set a scan range to +/−1.50D centering on −2.75D, and a step size at 0.5D. Therefore, the DCA 422 may be set to scan from −1.25D to −3.25D in −0.5D steps in this example to increasingly minus diopter powers.

The decision module 460 presents in an example embodiment a selected viewing target, e.g., a point source, for the patient to view, including a number of pixels at maximum or preset brightness intensity. The cluster of pixels preferably forms a round shape that appears as a point at a sufficiently far distance.

The test subject is ready to start, e.g., with an input device 440 in hand and with eyes aligned with an eyepiece of a self-service refraction instrument. The test subject's eyes are aligned along an optical path that includes corrective optics disposed between the test subject's eyes and the viewing target. The decision module 460 instructs the processor 410 to commence a scan to bring the point source viewing target stepwise to a more sharply focused line or point to the patient. The patient pushes a button or a trigger on an input device 440, to indicate the best image of a focused line has been reached as shown in FIG. 8A, as 810. The length of the line depends on the extent of the astigmatism in that patient. The patient may indicate an optimal image in other ways such as by releasing a button or verbal signaling or hand or face gesturing.

Next, a nesting method is used to pin point an improved value at the DCA. Based on this patient input, the decision module 460 may set, in an example embodiment, a scan range to +/−0.75D at the patient selected location at the DCA 422. For example, the patient may have picked −2.75 D. The new scan range may in an example embodiment now be −2.00D to −3.50D and the scan step size in this example may be reduced to −0.25D. This time, the patient may select −3.0D at the DCA 422. If a comparable refraction accuracy of a standard phoropter refraction procedure of 0.25 diopters is desired, one may stop here and record the sphere value for the patient. One may choose to continue to refine the accuracy to 0.125 D or even finer in a similar manner.

Each scan may involve in an example embodiment seven presentation positions at the DCA 422. At one second per step of the presentation in an example embodiment, the two scans will take about 15 seconds. The decision module 460 may decide to skip the first scan of 0.5 D step in an example embodiment if the decision module 460 is reasonably certain that the patient's refractive power has not changed beyond 0.75D.

At step 930 of FIG. 9, the decision module 460 may in an example embodiment decide to next find the astigmatism axis angle for the test subject. Again, the decision module 460 may decide to use the axis angle from an old eyeglasses prescription as a starting point. The decision module 460 may decide in an example embodiment to present a series of dots or multiple point sources forming a line, or multiple lines as in FIG. 8B. The decision module determines an angle of rotation of the line relative to the center based on an old eyeglasses axis angle of 135 degrees in this example embodiment. Now the patient sees a series of focused short lines, each centering at each of multiple points along a line pointing at 135 degrees. Example embodiments of how different test subjects may see the viewing target of FIG. 8B are provided at FIGS. 8C, 8D and 8E, and all angles are presented with the patient's perspective. The axis starts at the scientific minus x-axis, although viewed from the opposite direction, the axis may appear to start at the positive x-axis. In any case, the example of FIG. 8B shows two lines of point viewing targets 850, including one line rotated 135 degrees from the negative x-axis and the other line rotated 135 degrees from the positive x-axis. The lines of point viewing targets form an X shape on the display 450. This X shape may appear to a test subject with uncorrected vision imperfections as one of the examples provided at FIG. 8C, 8D, 8E or 8F may appear differently imperfect depending on the vision quality issues that the particular test subject may be dealing with.

Suppose that the actual cylinder axis of the patient is 150 degrees, not 135 degrees. Short focused lines at each of the dots will be pointing at 150 degrees, however, the centers of the short lines would be aligning along 135 degrees such as those shown in FIG. 8F. The decision module instructs the patient to rotate a knob on the input device 440 to effect a rotation of the sweep line formed by a series of dots. When the direction of the line is aligned with the short line exhibited by the astigmatism of the patient's vision, the short lines may or may not overlap depending on the spacings of the point viewing targets, but in any case can be made to become colinear and either form a solid line if the short lines overlap or a dashed line if the point viewing targets of FIG. 8B are not densely packed enough on the display 450 to cause overlap. One example of such a solid line is shown at FIG. 8C, while examples of such a dashed line are provided at FIGS. 8D and 8E. The test subject can easily fine tune the pointing direction by optimizing the line quality, with minimum or none of the short lines sticking out as they are in the test subject with uncorrected axis angle shown in FIG. 8F as being off by 15 degrees. Patient pushes a trigger to indicate the task done and the decision module 460 records the axis angle.

Note that the task of finding the angle of short lines in FIG. 8F is made considerably easier by using a series of points and aligning them to the direction of the short line. Moving the dotted line from 135 to 150 degrees by optimizing the quality of a line may take about 5 seconds. Returning to FIG. 9, the decision module 460 may decide to find round balls or otherwise circularly symmetric shapes at step 940. To neutralize astigmatism errors, the patient is asked to turn the short lines into symmetrical or round light balls. In other words, the lines shown in FIGS. 8C, 8D and 8E, are to become shorter and shorter and more and more similar to the point viewing targets of FIG. 8B when the astigmatism of each of these three test subjects is moved towards an optimally corrective amount.

Two cross lines of point viewing targets or short line viewing targets may be presented to a test subject. The two lines of points are pointing at 90 degrees cross, in one example, a first line is pointing at 135 degrees and a second line is perpendicular to the first line and is pointing at 45 degrees as illustrated at FIG. 8B. At each point source, there may be a short line as in the viewing target illustrated at FIG. 8A, except now there are multiple points forming two crossed lines, hence short lines arranged in a cross pattern.

The decision module 460 instructs the processor 420 to start an eye test scan using the cylinder value of the old eyeglasses −1.50D as a starting point, and setting the scan range to +/−0.75D in astigmatism diopter powers. In this example, the scan will cover from −0.75D to −2.25D in −0.25D steps. The test subject may see a shortening of focused lines initially, until the short lines turn into round, circularly symmetric balls or disks. If too much astigmatism is applied, short lines will begin to develop in the 90 degrees direction to the original short lines, i.e., in this example, the 135 degree short lines at FIGS. 8C, 8D and 8E will shrink to points or round disk shapes and then turn into short lines pointing to 45 degrees. This can be used as an indication that ACA is providing too much astigmatism correction.

At the optimal point, the image as perceived by the patient, includes the points arranged in an X shape just as the viewing target at FIG. 8B is actually presented. At this juncture, the test subject is asked to confirm that the task is done, and the decision module 460 records the astigmatism power. For example, the best astigmatism value may be at −1.25D. The time to scan through the astigmatism is about 7 seconds. Therefore, the entire refraction process can be done in less than 60 seconds.

The decision module 460 may decide to find the sharpest point at step 950 of FIG. 9. The decision module 460 may in an example embodiment keep cross lines of round viewing targets on the display 450 as viewing targets. The decision module 460 may present just a single point as in FIG. 8A. The DCA 422 is made to scan in −0.125D steps from the final position from Step 920 in FIG. 9. The test subject is expected to pick a DCA value when he or she sees the sharpest point when it is first formed. It should be at around half of the astigmatism value which is −0.625D. To avoid over minus power, the decision module 460 may limit the maximum DCA move to no more than −0.5 D beyond the projected value of −3.625D in an example embodiments. The decision module 460 may pick the less minus position selected by the test subject under repeated scanning at the DCA 422. For example, the final sharpest DCA value may be at −3.75D. Unless the decision module 460 decides to test and correct wavefront errors of the test subject's eyes, then the decision module 460 may decide that the self-service refraction procedure is completed and the optimized prescription for this patient is then illustrated at 960 of FIG. 9, which in the example embodiments described may be −3.125D sphere, −1.25D cylinder at 135 degrees.

### Controlling the Sensitivity of the Test Procedure

In a subjective refraction, the outcome derives from a combination of the eye's optics, retina function, visual pathway and the interpretive power of the brain. Defects in any one of the components can degrade the quality of vision. The preferred technique identifies the optimized vision in an efficient manner.

In one embodiment, the sensitivity of the eye test is controlled by the size and the brightness of the point source. The larger the light spot or the “point source”, the easier it is for the patient to identify end points. However, the larger the spot size, the worse is the spatial resolution and the attainable quality of vision. Therefore, an appropriate “point source” spot size is selected to attain a predetermined targeted level of quality of vision. One also controls the brightness of the presented point source to avoid saturation which may wash out details of starbursts and other features one may want to eliminate.

One may start with the assumption that 20/20 vision is achievable in a patient. One then sets the spot size to subtend a visual angle of 1 minute of arc. If the “point source” is placed at 6 meters from the patient, the object size is 1.75 mm for 1 minute of arc. One may use a slightly smaller spot size to account for diffraction and intensity saturation effects. For example, one selects a 1 mm diameter spot size point target at 6 meters for a 20/20 refraction.

If a patient undergoes the test through steps 1-4 above without too many repeated tests and delays, he or she is ready to move on to finding a better level of quality of vision. One may reduce the spot size to 0.5 mm at 6 meters for example and goes for the 20/10 vision.

On the other hand, a test subject with a cataract or macular degeneration conditions may have difficulty deciding the end points during the test steps 920-950, using a 1 mm target size and at 0.25D steps. One then increases the spot diameter to 1.5 or 2 mm or even larger until the patient can more easily identify the end points. When the spot diameter is increased to 2 mm or larger, the scan step size can also be increased from 0.25D to 0.5 D. The sensitivity reduction typically takes place at the step 920 or step 940 of FIG. 9. When the patient takes excessive amounts of time and/or cannot decide the end point after multiple presentations, the sensitivity is to be decreased, by increasing the spot size and increasing the step size as indicated in step 922 and step 942 of FIG. 9.

First, the steps 920-950 illustrated in FIG. 9 are to be completed assuming that the test subject shows no difficulty in finding all the end points. The decision module 460 may decide to generate a final prescription at 960 in an example embodiment.

However, if the patient has healthy eyes, after standard phoropter refraction he or she still sees starbursts forming around a single point that has been corrected for sphere, cylinder and axis, and he or she desires to attain better vision, one needs to correct the higher order aberrations of the eye. In another example embodiment, at step 970 of FIG. 9, the decision module 460 decides to increase the test sensitivity by reducing the spot size and the intensity of the light spot on the display 430, 450. The intensity of the point viewing targets 452 are set such that the test subject can see the starburst patterns around the point source and yet not too dim.

Next the decision module may set the scan step size to 0.01D for both DCA and ACA. The decision module may move the sphere correction to the start position at +0.25D from the final prescription after the completion of Step 4 above and at 950 of FIG. 9. The scan range may be set at +/−0.25D. In our example, the start positions will be −2.875D and the ACA set at −1.00D and scan towards more minus powers.

The decision module 460 next asks the test subject to find the most symmetric shape. Basically, this means repeating step 940 of FIG. 9, using a finer step size of 0.01D for example and a smaller size target of 0.5 mm diameter for example, at a non-saturating light intensity level. The test subject may use the input device to identify the location when the light source is most symmetrical or round. The decision module 460 may decide to repeat the scan to check the accuracy of the end point.

The decision module then asks the test subject to find the sharpest point. After ACA scan is completed, the decision module 460 may decide to scan the sphere power at DCA 422 in 0.01D steps, and repeat the scan to check accuracy of the end point. Steps 940 and 950 can be repeated until the test subject confirms that starbursts have been substantially reduced or eliminated.

The decision module 460 will determine whether the final prescription will be in the increments of 0.01 diopters in both sphere and cylinder, and whether the axis angle may be in 0.5 degrees increments, or whether different increments may apply depending on the sensitivities of the test process and the eyes of the test subject.

After all three optical components have been investigated one after the other in this example embodiment, the decision module may determine that it is time now to check the visual acuity of the test subject. One purpose for checking visual acuity here is to confirm whether any improvement has been accomplished by the refraction process. If the visual acuity level has reached a predetermined level which is considered acceptable as being the final refraction outcome, the refraction is completed at 360 in FIGS. 3A-3D. An example of a predetermined acceptable visual acuity level may be 20/20 or better, or equal to or better than 20/16, or 20/12. However, if the visual acuity is below the predetermined acceptable level, the decision module may continue the refraction, as indicated by the label 340 of the four example embodiments illustrated at FIGS. 3A-3D which brings the process to the decision module 460 and continues. Again, the decision module 460 decides at 330 of FIGS. 3A-3D which of the three optics components will be adjusted first, second, and third, as indicated above and as shown in FIGS. 3A-3D. The refraction process continues until the visual acuity reaches the predetermined acceptable level in visual acuity and finishes the refraction. In the event that the test subject's vision could not reach the acceptable level in visual acuity, the decision module 460 may decide to terminate the test after it has made multiple attempts or a limit of repeats is set at 2 times, 3 times, 4 times or another preselected number of times.

FIGS. 10A-10G illustrate seven images of a point-like viewing target as the point-like viewing target may appear to test subjects with certain uncorrected eye aberrations, or these seven images of a point-like viewing target may be seen by the same test subject at various stages of a process for generating a corrective optical prescription in accordance with example embodiments.

In the case of different test subjects with uncorrected eye aberrations, FIG. 10A illustrates a very blurry image of a point light source indicative of a large sphere error and potentially astigmatism that is in this case overwhelmed by the large sphere error. Reduction of the sphere error would reveal whether this test subject has any astigmatism issues.

FIG. 10B illustrates a blurry image that is clearly elongated such that the test subject has a modest sphere issue and an astigmatism issue.

FIGS. 10C and 10F illustrates substantially focused linear images indicating that these test subjects only appear to have astigmatism issues, one test subject having a larger astigmatism error than the other, such that the decision module 460 may decide to efficiently test and correct only astigmatism and not sphere in both of these cases.

FIG. 10D illustrates a blurred round or slightly oblong shape indicating that the test subject has a modest sphere issue and a slight astigmatism issue, such that the decision module 460 would test and correct the sphere issue, but may or may not decide to test and correct astigmatism issue. Once the sphere is corrected, the decision module 460 will be better able to determine whether to test and correct astigmatism, particularly if the test subject indicates a desire either to test or not to test and correct for the slight astigmatism that the test subject appears to have.

FIGS. 10E and 10G illustrate symmetric shapes that are mostly clear point-like images, such that a test subject with vision this good might not need a vision test. However, a self-service refraction instrument in accordance with an example embodiment may be able to improve the test subject's visual acuity from the image shown at FIG. 10E to the image shown at FIG. 10G, which may improve the test subject's visual acuity from 20/20 to 20/12 or better.

In the case of a same test subject at different stages of a self-service refraction test process, FIG. 10A illustrates an initially very blurry image of a point light source. The decision module 460 would likely decide to correct the sphere error first initially at a large step size until the test subject starts to see something like the elongated blurry image of FIG. 10B. The decision module 460 could choose to continue to correct the sphere error until the test subject sees something like the image of FIG. 10C, and then correct astigmatism to arrive at the image of FIG. 10E or the decision module 460 may decide to correct astigmatism until the test subject sees something like the image of FIG. 10D, and then decide to correct sphere again to arrive at the image of FIG. 10E. In this latter case, wherein the decision module 460 decides to correct astigmatism to go from the image of FIG. 10B to the image of FIG. 10D, a second sphere correction sub-routine may reveal a small but significant astigmatism such that the decision module 460 may decide that a second astigmatism correction sub-routine should be undertaken to ultimately arrive at FIG. 10G. In the former case, wherein the decision module 460 decides to continue to correct sphere to go from the image of FIG. 10B to the image of FIG. 10C, and then to correct astigmatism to arrive at the image of FIG. 10E, the decision module 460 may decide to continue to test and correct sphere until the image appearing to this test subject is in accordance with FIG. 10G. In this case, the additional sphere correction may have been determined by the decision module 460 as being likely to achieve and significant, such that the test subject may have gotten a prescription correcting her vision to 20/12 from 20/20 with the last sphere correction routine.

The results of a self service refraction eye test in accordance with certain embodiments may be provided to a physician or optometrist to review in a condensed form, for example, as described by the present inventor/applicant at United States patent application publication number 2020/0037868, entitled “Concise Representation for Review of a Subjective Refraction test,” which is incorporated by reference. The physician or optometrist advantageously could serve more patients because reviewing the results of the self service refraction tests in condensed representation in accordance with certain embodiments takes less of the physician's time per patient than performing each test side by side with each test subject start to finish.

Optionally, a quasi self service refraction test may be performed primarily by the test subject at a quasi self service kiosk with the supervision of a trained expert technician or optometrist being available to assist, in person or through a live chat or online telemedicine module that may be built into the quasi self service kiosk, or the expert may be available by downloading a separate app onto the test subject's mobile device, as needed. In this example embodiment, the expert may be assigned to supervise multiple quasi self service kiosks simultaneously, and may be available to assist any of several test subjects who are primarily running their own self-service eye examinations at different, proximate and/or remote, quasi-self service kiosks. Such technician may not need to be an expert optometrist or physician, but may have familiarity with the operation of the quasi self service kiosk or otherwise have a training background far less stringent than that required of a physician, optometrist or expert technician, and online support or telephone support, or a guide manual, text, audio and/or video, or other help center support may be available that is sufficient to help a test subject during an user-friendly self service refraction eye examination.

### Head Tilt Angle Monitoring

An apparatus is provided for monitoring a tilt angle of a patient's head during an ophthalmic measurement, such as a self-service refraction test by decision module in accordance with embodiments described above herein. In one example embodiment, a head tilt angle monitoring apparatus includes an electronic level sensor configured to acquire tilt angle data of a patient's head during an ophthalmic measurement. A head band, glasses, a hat, headphones, one or more ear clips, or other head gear, or combinations thereof, has the electronic level sensor coupled therewith and is configured to couple in a fixed orientation relative to an orientation of the patient's head. A communication circuit is configured to export the tilt angle data acquired by said electronic level sensor to an angle indicator.

The head band, glasses, hat, headphones, one or more ear clips, or other head gear, or combinations thereof, may be coupled to or integrated with a head phone device that is configured to produce an audio angle indicator.

The angle indicator may include (i) a computer tablet or other mobile computing device that includes a real-time digital display of said tilt angle data streaming from said electronic level sensor, or (ii) an audio speaker, ear bud, headphone device or other audio transmission device configured to provide one or more text to voice messages or other sounds in accordance with said tilt angle data streaming from said electronic level sensor, or (iii) a mobile device configured to generate text or voice message announcements at a mobile device of the patient, or (iv) combinations of these.

The communication circuit may include (i) Bluetooth or (ii) other wireless antenna or transmitter, or (iii) a hard wired or plug-in electrical connection to the angle indicator, or (iv) combinations of these.

A reference leveling device may be configured to determine an angular offset reference point to a true “vertical” orientation. In certain embodiments, when the reference leveling device indicates that it is disposed precisely horizontally, then the eyes of the patient will be disposed at a same relative height, such that neither the patient's right eye nor left eye is closer to the ground than the other. A line drawn between the centers of the patient's pupils may be approximately parallel to the long dimension of the level such that when the level determines that it is oriented horizontally then the patient's head can be relied upon to be within an acceptable tolerance deviation angle from a 0° tilt angle or upright orientation or portrait orientation. The reference leveling device may include one or more sensors that are angled at a predetermined angle to the line drawn between the patient's two pupils, wherein the predetermined angle is deemed to be a threshold at which an alarm or other communication is consciously perceivable by the self-service refraction test subject, or in an assisted refraction test by a physician, optometrist, technician, automated testing device and/or the patient herself or himself so that the patient can return to a 0° tilt angle, upright and/or portrait orientation.

In accordance with an example embodiments, FIG. 11 illustrates schematically a self-service subjective refraction test subject wearing head gear 1110 that includes an electronic level sensor 1120 coupled to or integrated with the head gear 1110 being worn by the test subject 1100. The decision module 460 of FIG. 4 and/or the self-service refraction test subject herself may be informed by head tilt angle measurement data whenever the head of the test subject 1100 has become tilted more than a threshold amount during an eye measurement procedure. The test subject 1100 may be additionally or alternatively informed automatically that a threshold head tilt angle has been breached, e.g., in ear phones 1130 or through a speaker (not shown in FIG. 11) on the head set 1110, or on another device such as the self-service refraction device 1140, or on a mobile device 1150 after being communicated wirelessly from the head set 1110.

In certain embodiments, an alarm or other warning sound may include an alarm or noise or any melody or rhythmic audio clip including any number of one or more beats that may repeat at certain intervals, until corrective action is taken by the test subject 1100, e.g., to sit up straight with or without assistance to level the head. The sound may get progressively louder for as long as it takes to incentivize the test subject 1100 to take corrective action rapidly to preserve the quality and integrity of the eye measurement to achieve useful results in the form of an accurate prescription for lenses or a lasik pattern, for non-surgical eye tissue correction or for a surgical eye correction procedure. The alarm may alternatively or additionally include a vibration and/or a strobe light and/or another change in lighting or background that is easily and quickly noticeable by the test subject, even if her eyes are partly or wholly closed at the time or focused on a viewing target provided by the self-service refraction device 1140.

An eye exam which utilizes subjective and/or objective refraction techniques can be completed with reliable assurance that the test subject's head was not tilted more than the predetermined angle during the test other than perhaps for a fraction of a second about the same time that the reference level sensor determined that the angularly offset level sensor component was deemed to be parallel with horizontal. The reference leveling device may include (i) a bubble level, (ii) a laser level, (iii) a box level, (iv) a torpedo level, (v) an I-beam level, (vi) an optical level, (vii) a surveyor's level, (viii) a cross-check level or a bulls-eye level for determining that the patient's head is level with a 2D horizontal plane, or (ix) a digital or analog electronic level, (x) a pre-calibrated level device, or (iv) combinations of these or other types of levels known to those skilled experts in the use of levels.

Examples of reference leveling devices that may be coupled with a wearable earphone, earbud, head phone, helmet, hat head band, eyeglass frame, denture, tooth alignment or brace device or other dental device, wig or hair piece, tie, collar, necklace, chin rest or chin strap, respiratory filter, cloth mask, or oxygen mask, goggles, neck brace or other wearable device, include the Tacklife™ MT-LO3 12 inch level, the MD 92379 Smarttool Rail or 92500 SmartTool Gen3 Digital Level, Gummerson Tools Inclinometer or Digital Level Protractor Angle Finder, Zircon Ultra level pro, Accusize 8″ master precision level serial number #5908-C685 which may have an accuracy between 0.02″/10″ and 0.0002″/10″ or between 0.002″/10″ and 0.0002″/10″, the Tacklife MDP02 advanced digital protractor level/bevel gauge/angle gauge, Shefio's IP54 dust and waterproof electronic level tool, the Checkpoint 0300PL Pro mag precession torpedo level, Tacklife Spirit level, DOWELL 9 inch magnetic box level torpedo level which may have three different bubbles, e.g., 45°, 90°, 180° or 0°, 10°, 20°, M-D Building Products 92346 SmartTool Module, TekcoPlus angle finder ruler tool gauge and/or long digital inclinometer protractor with or without magnetic base, GemRed Digityal level Angle Slope Level, Hammerhead Digital Level with Laser, Semloss multipurpose laser level measuring tape standard and metric tape ruler, AdirPro 32x Optical Auto Level self leveling tool, Qooltek multipurpose laser level laser measure line, Stabila 29124 type 80A-2 measuring stick level, Rack-A-tiers 45404 Jet Level, Stabila 36514 type 196-2 tech 14′ level, Fitmaker angulizer ruler template tool, General Tools 828 Digital Sliding T-bevel gauge and digital protractor, PLS180 Red Cross L:ine Laser Level PLS-60521N by Pacific Laser Systems, Bosch Self-Leveling Cross Line Laser GLL-55, GLL 30, or GLL 3-80, Huepar 901CG or 902CG or box 1G professional laser level mute 150 ft. green beam cross laser self-leveling alignment tool, Goldblatt 3-piece Torpedo Spirit Level Set, Suaoki P7, Workpro WO02901A 4-piece measuring tool set, torpedo, spirit level, GemRed Digital Level angle slope (N0.420 digital torpedo level with or without magnets), Brillife laser leveler spirit level line lasers ruler, gradienter horizontal ruler, Kole GW323 multi-purpose laser level with or without suction mount, Pacific Laser Systems PLS 180, Navegando 5 line 6 point 360° rotary multipurpose self-leveling output 4 vertical, Johnson level and tool 40-6648 self leveling cross and line laser, URCERI 9211R line and plumb self-leveling horizontal, vertical cross line, YOUTHINK laser measuring device with Pythagorean mode, measure distance, area and volume, Leico disto e7500i, DEWALT DW03050 laser distance measurer, LESHP Handheld laser distance measure, Perfect-Prime RF0350 Diastimeter, and/or Fnova splash and dust proof distance measure, or combinations of two or more thereof.

The tilt angle data may include an angular offset reference to a true vertical orientation, such that the angle indicator receives or communicates, or both, a deviation angle from the true vertical orientation.

A warning alarm circuit or app may be configured to trigger communication of a warning alarm signal when the tilt angle data indicates a deviation angle from the true vertical orientation that exceeds a predetermined angular limit.

In an embodiment configured so that a test subject 1100 may self-correct his or her head orientation embodiment, the angle indicator may be configured to receive tilt angle data and to communicate to the patient in real time a beep sound, or a voice message announcement or other audio, visual or other sensory warning signal when the tilt angle data indicates a deviation angle from a true vertical orientation that exceeds a predetermined angular limit. A feedback mechanism may be configured to guide the test subject to correct his or her head tilt angle, in response to the warning signal, back to an optimum head alignment position during an self-service eye measurement procedure.

An angle indicator may receive and/or communicate tilt angle data calibrated to an angular offset from a true vertical orientation of the test subject's head. In another embodiment that is configured so that a test subject may self-correct his or her head orientation, the angle indicator may be configured to receive tilt angle data and to communicate to the test subject in real time a beep sound, or a voice message announcement or other audio, visual or other sensory warning signal when the tilt angle data indicates that a deviation angle from an angular offset orientation exceeds a predetermined angular limit, wherein the angular offset orientation may differ by the angular offset from the true vertical orientation. In this embodiment, a feedback mechanism may be configured to guide the test subject 1100 to correct his or her head tilt angle in response to the warning signal back to the angular offset orientation as an optimum head alignment position during an eye measurement procedure.

A method of monitoring a tilt angle of a patient's head during an ophthalmic measurement is also provided. The method includes coupling an electronic level sensor 1120 in a fixed orientation relative to an orientation of a test subject's head. Tilt angle data of the test subject's head may be acquired during an ophthalmic measurement in an example embodiment. The tilt angle data acquired by an electronic level sensor 1120 coupled to head gear 1110 may be exported to an angle indicator in an example embodiment.

The electronic level sensor 1120 may be coupled to a head band, glasses, hat, headphones, one or more ear clips, a face shield or face mask, or other head gear, or combinations of, that may be coupled to a patient's head during an eye measurement. The electronic level sensor 1120 may be coupled onto or integrated with a head phone device that is configured to produce an audio angle indicator.

The exporting of the tilt angle data to the angle indicator may include streaming the tilt angle data from the electronic level sensor 1120 to the angle indicator.

The exporting of the tilt angle data to the angle indicator may include Bluetooth or other wireless transmission or transmission over a hard-wired or plug-in electrical connection, or combinations of these.

An angular offset reference point to a true vertical orientation may be determined. The determining of the angular offset reference point may involve utilizing (i) a bubble level, (ii) a laser level, or (iii) a pre-calibrated level device, or (iv) combinations of these.

The tilt angle data may include an angular offset reference to a true vertical orientation, such that the exporting of the tilt angle data to the angle indicator may include exporting the deviation angle from the true vertical orientation along with the tilt angle data and/or exporting deviation angle-adjusted tilt angle data.

The method may include triggering a warning alarm signal when the tilt angle data indicates a deviation angle from the true vertical orientation that exceeds a predetermined angular limit. The method may include communicating to the patient in real time a beep sound, or a voice message announcement or other audio, visual or other sensory warning signal when the tilt angle data indicates a deviation angle from a true vertical orientation that exceeds a predetermined angular limit. The method may further include guiding the patient to correct his or her head tilt angle in response to the warning signal back to an optimum head alignment position during an eye measurement procedure.

The method may include indicating the tilt angle in real time during an eye measurement. The indicating of the tilt angle in real time may include (i) displaying said tilt angle on a computer tablet or other mobile computing device, or (ii) generating one or more text or voice messages or other sounds in accordance with said tilt angle data at an audio speaker, ear bud, headphone device or other audio transmission device, or (iii) generating on a mobile device of the patient text or voice message announcements, or (iv) combinations of these. The indicating may include receiving and/or communicating tilt angle data calibrated to an angular offset from a true vertical orientation of the patient's head.

The method may include communicating to the patient in real time a beep sound, or a voice message announcement or other audio, visual or other sensory warning signal when the tilt angle data indicates that a deviation angle from an angular offset orientation exceeds a predetermined angular limit, wherein the angular offset orientation may differ by the angular offset from a true vertical orientation. The method may include guiding the patient to correct his or her head tilt angle in response to one or more further sensory signals back to the angular offset orientation as an optimum head alignment position during an eye measurement procedure.

Another method and apparatus are provided that are configured for head orientation self-correction by an eye measurement patient during an eye measurement. In the method, a tilt angle of a patient's head is monitored during an eye measurement procedure. A beep sound or a voice message or other audio, visual, vibratory or other sensory warning signal is generated and communicated to the patient during the eye measurement warning the patient that his or her head is tilting at least a certain predetermined angular limit amount. One or more further sensory signals and produced and communicated to the patient to guide the patient to correct his or her head orientation and thereby maintain his or her head orientation within said certain predetermined angular limit amount during the eye measurement.

The method may involve generating and communicating a sensory warning signal that includes controlling sound volume based on amount of head tilt angle. The controlling of the sound volume may include increasing sound volume as head orientation deviates farther from true vertical orientation or from an offset orientation which was set as a reference point.

The method may involve generating and communicating a sensory warning signal that includes controlling a beep frequency based on amount of head tilt angle. The controlling of the beep frequency may include increasing the beep frequency as the head orientation deviates farther from true vertical orientation or from an offset orientation which was set as a reference point.

The method may involve generating and communicating a sensory warning signal that includes controlling an intensity of a visual and/or vibratory warning signal based on amount of head tilt angle. The controlling of the visual and/or vibratory intensity may include increasing sound intensity as head orientation deviates farther from a true vertical orientation or from an offset orientation which was set as a reference point.

In an apparatus, means for patient self-correction of head orientation during an eye measurement are provided. The apparatus includes means for monitoring a tilt angle of the patient's head during an eye measurement procedure; means for generating a beep sound or a voice message or other audio, visual, vibratory or other sensory warning signal to the patient during said eye measurement warning the patient that her head is tilting at least a certain predetermined angular limit amount; and means for producing one or more further sensory signals to the patient to guide the patient to correct her head orientation and thereby maintain her head orientation within said certain predetermined angular limit amount of tilting of her head during the eye measurement.

The apparatus may include means for generating and communicating a sensory warning signal that includes means for controlling sound volume based on amount of head tilt angle. The means for controlling sound volume may include means for increasing sound volume as head orientation deviates farther from true vertical orientation or an offset orientation which was set as a reference point.

The apparatus may include means for generating and communicating a sensory warning signal that includes means for controlling a beep frequency based on amount of head tilt angle. The means for controlling beep frequency may include means for increasing the beep frequency as the head orientation deviates farther from true vertical orientation or from an offset orientation which was set as a reference point.

The apparatus may include means for generating and communicating a sensory warning signal that includes means for controlling an intensity of a visual or vibratory warning signal, or both, based on amount of head tilt angle. The means for controlling an intensity of a visual and/or vibratory warning signal may include means for increasing sound intensity as head orientation deviates farther from true vertical orientation or from an offset orientation which was set as a reference point.

The eye has certain muscles that cause the eye to move side to side in horizontal direction and up and down direction without moving the head. However, there is no muscle in the eye that may cause the eye to rotate along the optical axis of the eye. Hence vision correction using eyeglasses is based on a stationary position of the glasses resting on patient's nose and with temples resting over the patient's ears. Likewise, a refractive surgery makes refractive power changes on the cornea or in the stroma of the eye, as in LASIK, or PRK or corneal on lay or inlay procedures. Whether it is a vision correction by eyeglasses, or by laser surgery, the corrective powers are determined prior to the making of the corrective lenses in the case of eyeglasses, or prior to applying the laser tissue ablation pattern to the eye. Multiple example embodiments are provided of refraction measurement methods and equipment that are configured such that the axis angle of the astigmatic correction is reliably aligned with the axis of a corrective tool, such as a pair of glasses, or an ablation pattern orientation provided by a refractive surgery. Therefore if the patient's eye or head is tilted during an axis measurement, such as a refraction measurement, undesirable residual refractive errors may likely result from the treatment.

An electronic level sensor may include an accelerometer, a gyrometer, a bubble level, a laser level, or an optical face or head orientation device including a camera, a processor and a software application for determining a face or head orientation of an eye patient and/or changes in face or head orientation.

Head gear can include anything that may be configured to couple to an eye patient's head and/or to couple in fixed relative orientation with an eye patient's head such as attachment at the top of the neck or an unattached or quasi-attached device that responds in known proportion to changes in an eye patient's head orientation.

Communication circuits may include electronic, magnetic and/or optical components, digital or analog, with or without a processor and/or software app that may generate and/or communicate signals that include tilt angle data and/or data that is representative of the tilt angle data. Communication circuits or one or more components thereof may include an angle indicator or one or more components thereof.

An angle indicator may communicate or relay a sensory signal to an eye patient that includes an audio, visual, and/or touch sensory signal, and/or even a smell or taste signal and/or a cognitive signal that includes tilt angle data and/or data that is representative of the tilt angle data. An angle indicator may or may not receive tilt angle data and may or may not communicate tilt angle data and may communicate to the patient, another person and/or a machine capable of effecting real time patient head tilt angle correction.

### Optical Assemblies for Defocus and Astigmatism Correction

FIG. 12 schematically illustrates an optical assembly for a self-service refraction instrument in accordance with an example embodiment. An astigmatism corrector assembly 1230 or ACA 1230 is located approximately at the spectacle plane 1240 near the eye of the test subject. The ACA 1230 may comprise a pair of lenses 1231 and 1232 which may be rotated independently so that astigmatism power is adjustable by relative rotation of lenses 1231 and 1232, and astigmatism axis angle may be adjusted by synchronous rotation of lenses 1231 and 1232 relative to an orientation of the test subject and other optics within the optical assembly. A defocus corrector assembly 1220 or DCA 1220 is disposed further away from the eye of the test subject along an optical path along which the test subject's eye is aligned for observing a viewing target 1211 on a display 1212. The DCA 1220 includes a pair of lenses f1 1221 and f2 1222, wherein lens f1 1221 is disposed between the ACA 1230 and the lens f2 1222. The lens f2 1222 is translatable along the optical path forward and backward of the optical assembly in this example embodiment. In an alternative embodiment, the lens f1 1221 is movable. One or both of the lenses f1 1221 and f2 1222 may be translatable along the optical path to adjust sphere power by increasing or decreasing a spacing between lens f1 1221 and lens f2 1222 of the DCA 1220. A waveplate 1224 is shown disposed in the conjugate pupil plane of the test subject's eye under examination in the example embodiment illustrated schematically at FIG. 12, while other example embodiments include just an ACA 1230 and a DCA 1220. The waveplate 1224 is configured in this example embodiment for correcting higher order aberrations such as coma, or to eliminate starburst aberrations using corrective optics.

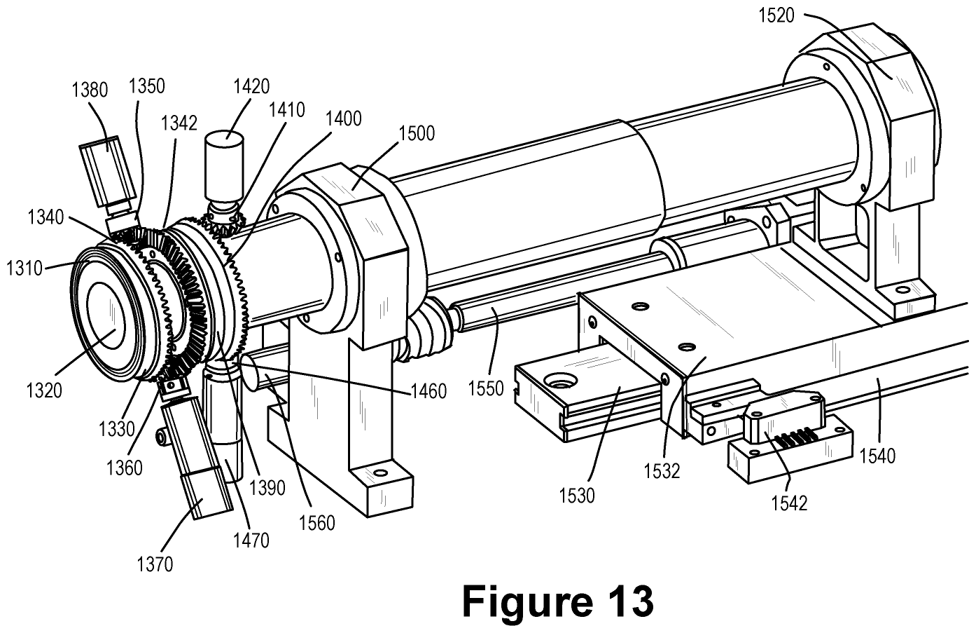

In accordance with another example embodiment, a motorized mechanism is provided for adjusting an optical assembly automatically in a self-service refraction test in accordance with an example embodiment. Referring now to instrument that is schematically illustrated at FIG. 13, pairs of lenses or Zernike wave plates are mounted on a rotary ball bearing. One of the lens pairs 1320 is mounted on rotary ball bearing 1310. A view of the second ball bearing and of the second pair of lens or Zernike wave plates is blocked in FIG. 13 by the housing of the instrument, while the mounting and the motion mechanism is similar to that of the first wave plate. The inner ring of the ball bearing 1310 is attached to a bevel gear 1340. Similarly, the second wave plate is mounted with a second ball bearing, and the inner portion of the second ball bearing is attached to a second bevel gear 1342.

In one embodiment, two pinion gears are used. One pinion gear 1360 may be used to drive both bevel gears, as illustrated in FIG. 13, which in turn rotate the pair of wave plates 1320 in equal angles but in opposite direction. This counter rotating motion of the Zernike wave plate pair 1320 accomplishes the goal of adjusting the amplitude value of the combined wave plate assembly. When two identical wave plates of a selected Zernike function are substantially aligned with overlapping optical axes, the paired wave plates generate the maximum wavefront amplitude. Using trefoil as an example, if one uses two wave plates, each with amplitude of 2.5, the maximum amplitude achievable is the sum, or 5. As the relative angle between the wave plate pair increases, the overall amplitude of the assembly unit decreases, and the sum amplitude becomes zero when the optical axis of the two trefoils are at 90/3, or 30 degrees apart. Therefore, an amplitude control ranges from zero to a maximum of 5. Other desirable adjustable ranges may be constructed similarly utilizing two identical wave plates each having half of a desired maximum.

A motor unit 1370 illustrated in FIG. 13 is attached to a drive pinion gear 1360. The motor unit 1370 can include a DC motor, a step motor, or another suitable mechanism that turns the pinion gear 1360. A second pinion gear 1350 is also mounted between the two bevel gears 1340 and 1342. This second pinion gear is used as a rotary angle sensing device and is attached to a rotary encoder 1380. Electrical output is fed to an encoder reader which reads pulses and pulse edges. This information is converted to an angular position of the optical axis of each of the wave plates. A second computer program routine then calculates the sum amplitudes of the two wave plates, from the relative angular movement for a given amplitude of the individual wave plates. An overall amplitude of the wave plate pair is then displayed in a monitor, LED, LCD, or any suitable display device.

Outer rings of ball bearings 1330 and a corresponding outer ring for the second ball bearing are attached to inner rings of third and fourth ball bearings. The outer rings of the third and the fourth ball bearings are in turn supported and mounted to the base of the instrument (not shown). The outer ring of the fourth bearing 1390 is shown in FIG. 13, but the view of its inner ring is obscured. That inner ring of the fourth ball bearing 1390 is attached to a third bevel gear 1400. The entire counter rotating unit of the first and second bearings are affixed to the inner ring of the fourth bearing 1390, and a second motor 1470 is connected to and drives the third pinion gear 1460 in this example embodiment, which in turn rotates the entire counter rotating assembly comprising the first and second ball bearings and the counter rotating wave plates. A second rotary encoder 1420 is attached to a fourth pinion gear 1410 and senses an angular rotation of the entire counter rotating assembly, which is the angle φ of the optical axis of the entire counter-rotating wave plate pair. Again, the electrical output of the encoder 1420 is fed to an encoder reader. A separate computer routine converts electrical pulses from the encoder into an angle reading, which is the angular orientation φ of the optical axis of the wave plate pair.

Instead of using pinion gears to drive the two wave plates of the ACA 1320, which are preferably substantially identical, in opposite direction, at identical angular rates or otherwise in identical angular amounts per increment, one may use synchronized motor drives. In such construction, each wave plate is driven by its own driver electronics. However, two driver circuits are controlled by a closed loop algorithm, such that the two motors still move substantially in “lock-step”, or move continuously or jog in steps, in substantially identical angle increments in the same or opposite directions, during any commanded movement. The motor movement is monitored by rotatory encoder. An amplitude precision greater than 0.01 diopters is in this way achievable in a 6 diopter astigmatism adjustable wave plate unit.

Indeed, FIG. 13 illustrates an optical assembly that includes two continuously adjustable Zernike functions, i.e., the astigmatism and the defocus. The astigmatism wave plate assembly is shown on the left in FIG. 13, and is also referred to as an illustration of how other continuously adjustable wave plate assemblies may be constructed. The defocus assembly includes two lenses f1 (1221 in FIG. 12) and f2 (1222 in FIG. 12), which are mounted in optics holders 1510 and 1520, respectively, in the example embodiment that is schematically illustrated in FIG. 13. The lens mount 1520 is affixed on a linear slide 1530, which is movable along the optical axis of the patient's line of sight for adjusting defocus power. A linear encoder strip 1540 is attached to the movable platform 1532, and an encoder reader head 1542, generates electrical pulses as the encoder strip travels across it. The encoder output is fed to a pulse counter, and the processor 410 converts the count into the location of lens f2 (1221 in FIG. 12) relative to lens f1 (1222 in FIG. 12), and the processor 410 subsequently calculates the diopter power of the defocus assembly unit. The diopter reading is accessible to the decision module 460 for use in deciding next steps in the self-service refraction process. The movable platform of the linear slide is driven by a lead screw 1550, which is turned by a motor 1560. Any kind of motor with the desired speed, resolution and accuracy may be used.

Equipment and methods for performing a refractive eye measurement and/or for determining and/or correcting aberrations of human eyes that may be combined within alternative and/or additional embodiments are described at U.S. Pat. Nos. 6,706,036, 6,325,792, 6,761,454, 9,408,533, 9,320,426, 9,247,871, 8,684,527, 8,632,183, 8,632,184, 8,950,865, 8,967,801, 8,790,104, 8,409,177, 8,388,137, 8,366,274, 8,262,220, 8,113,658, 8,033,664, 7,954,950, 7,909,461, 7,824,033, 7,699,471, 7,726,811, 7,695,134, 7,490,940, 7,461,938, 7,425,067, 7,293,871, 7,220,255, 7,114,808, 5,984,916 and 5,549,632, and at US published application no. 2005/0174535, and each of these patents and published patent applications is hereby incorporated by reference.

While an exemplary drawings and specific embodiments of the present invention have been described and illustrated, it is to be understood that that the scope of the present invention is not to be limited to the particular embodiments discussed. Thus, the embodiments shall be regarded as illustrative rather than restrictive, and it should be understood that variations may be made in those embodiments by workers skilled in the arts without departing from the scope of the present invention.

In addition, in methods that may be performed according to preferred embodiments herein and that may have been described above, the operations have been described in selected typographical sequences. However, the sequences have been selected and so ordered for typographical convenience and are not intended to imply any particular order for performing the operations, except for those where a particular order may be expressly set forth or where those of ordinary skill in the art may deem a particular order to be necessary.

A group of items linked with the conjunction “and” in the above specification should not be read as requiring that each and every one of those items be present in the grouping in accordance with all embodiments of that grouping, as various embodiments will have one or more of those elements replaced with one or more others. Furthermore, although items, elements or components of the invention may be described or claimed in the singular, the plural is contemplated to be within the scope thereof unless limitation to the singular is explicitly stated or clearly understood as necessary by those of ordinary skill in the art.

The presence of broadening words and phrases such as “one or more,” “at least,” “but not limited to” or other such as phrases in some instances shall not be read to mean that the narrower case is intended or required in instances where such broadening phrases may be absent. The use of the term “apparatus” does not imply that the components or functionality described or claimed as part of the assembly are all configured in a common package. Indeed, any or all of the various components of an apparatus, e.g., head gear and angle indicator may be combined in a single package or separately maintained and may further be manufactured, assembled or distributed at or through multiple locations.

1. A self service refraction eye test method, comprising
a. initiating a self-service refraction procedure when a test subject activates a first user interface input device that is configured to communicate with an on-board processor of a self-service refraction instrument;
b. aligning one or both eyes of the test subject along an optical path that includes corrective optics disposed between said one or both eyes of the test subject and a viewing target, including indicating by communicating to the test subject a relative position and orientation of said one or both eyes of said test subject to a preset alignment range;
c. programming the processor of the self-service refraction instrument including instructing the processor to facilitate a self-service subjective refraction eye test, including adjusting specific choices of values and step size adjustments of values of selected optical parameters of corrective optics, including sphere, cylinder or axis or higher order aberrations or combinations thereof; based on subjective input from the test subject and an expert database of refraction rules;
d. presenting said choices of corrective optics through which the test subject can see one or more viewing targets more or less clearly or with one or more correctable optical aberrations of sphere, cylinder or axis or higher order aberrations of one or both eyes of the test subject, or combinations thereof;
e. receiving communications from the test subject indicating which of the choices appears more or less clear or blurry to the test subject;
f. repeating the adjusting and presenting of different choices of corrective optics, or checking visual acuity; or both; based on the communications from the test subject indicating said choices; and
g. generating; through iterating above steps c, d and e, and checking visual acuity and referencing said expert database of refraction rules, a corrective optical prescription for said one or both eyes of the test subject to improve vision quality of the test subject to better than a target visual acuity.

2. The self-service refraction method of claim 1, comprising presenting at least two choices of images of a viewing target to the test subject using different configurations of an optical assembly placed in a line of sight of the test subject and through which the viewing target appears more or less clearly to the test subject, and wherein the viewing target comprises an eye chart, a PSF target, a still photo or a live scenery or combinations thereof.

3. The self-service refraction method of claim 1, comprising deciding next steps during the adjusting and presenting of said different choices of corrective optics based on learned statistical probabilities or programmed rules, or both, for improving the visual acuity of the test subject.

4. The self-service refraction method of claim 1, comprising deciding whether to present another choice of corrective optics, or instead to test a visual acuity of the test subject using a current choice of corrective optics, as a next step in the self-service refraction process.

5. The self-service refraction method of claim 1, comprising deciding which of sphere, cylinder and axis optical components will be adjusted next.

6. The self-service refraction method of claim 1, comprising deciding how much to adjust a step size of an optical component for presenting a next corrective optics choice to the test subject.

7. The self-service refraction method of claim 1, comprising deciding when an optical component has been adjusted to its optimal value, such that no further meaningful improvement is likely nor is to be attempted by further testing.

8. The self-service refraction method of claim 1, comprising checking visual acuity to confirm whether an improvement in visual acuity has been made by adjusting one or more values of the corrective optics prescription.

9. The self service refraction method of claim 1, comprising continuing said repeating of steps c, d, e and f until a visual acuity test score has not improved by a preset meaningful amount compared with a previous visual acuity test score.

10. The self-service refraction method of claim 1, comprising terminating the self-service refraction process when a visual acuity score has reached or exceeded a preset acceptable visual acuity score level.

11. A self-service refraction eye test apparatus, comprising:
a. an instrument housing having a window defined therein through which a test subject can view a viewing target on a display;
b. a corrective optical assembly coupled within the instrument housing between the window and the display, comprising
(i) a defocus corrector assembly including a pair of adjustably spaced apart lens elements and
(ii) an astigmatism corrector assembly including a pair of independently rotatable lens elements;
c. one or more motors coupled to one or more adjustable optics of the optical assembly;
d. a motion control unit signal coupled to the one or more motors for controlling movements of the one or more adjustable optics of the corrective optics assembly;
e. a processor;
f. one or more digital storage media having code embedded therein including a decision module informed by a refraction rules database configured to provide decisions regarding next steps in a self-service refraction process, and based on decisions provided by the decision module; the processor instructs the motion control unit to adjust the optical assembly by moving one or more of said adjustable optics and presenting a further choice of corrective optics to the test subject; and
g. a test subject input device for actuation by the test subject to communicate choices between pairs of corrective optics through which the one or more viewing targets presented to the test subject are indicated by the test subject as appearing more or less clear or optically, aberrant.

12.-73. (canceled)

74. A self service refraction device, comprising;
a. a computer or a similar electronic device, comprising a processor and software code embedded in a digital storage medium for programming the processor to perform a self-service refraction test involving untrained users without a trained technician present; one or more human interface devices including one or more of a mouse, a joystick, a touch screen, a dial control, and an audio input and output; a camera configured to capture eye images; wherein the software code is configured for programming the processor to execute a self-service refraction process, including taking input from the one or more human interface devices, audio input, and camera input, and
b. a software code embedded in a same or separate processor-readable storage medium for providing guidance to the user; using a computer-generated voice, or pre-recorded human voice instructions for the user to operate the self-service refraction device without prior training;
c. an eye chart code embedded in a same or separate processor readable storage medium for displaying to the user to read in performing a Visual Acuity test;
d. a Decision Module comprising non-transitory executable code embedded in a same or separate processor readable storage medium for making decisions during a self-service refraction test based on a set of rules embedded in a readily accessible same or separate processor-readable storage medium and directing the refraction device and refraction processes in accordance with deciding one or more of the following:
i. which of the three optical components should be adjusted next;
ii. how big a step size of change to use in presenting viewing targets through choices of optics with differing values of an optical component;
iii. when does said optical component reach its optimal point, at which optimal point no further meaningful improvement should be attempted, and at which optimal point to end said presenting viewing targets through further choices of optics with differing values of said optical component;
iv. when to check Visual Acuity score to confirm improvement has been made;
v. when to repeat steps i. to iv. until the Visual Acuity score has not changed by a meaningful range, wherein the preset meaningful range is defined by an improvement of less than a number of lines or letters in a Snellen eye chart or other symbols in another VA test eye chart;
vi. when to end the refraction process when the Visual Acuity is hovering within the meaningful range;
e. an optics assembly comprising adjustable optics components capable of changing the sphere, cylinder and axis power of various configurations of said optical assembly through which viewing targets are presented to the user;
f. a viewing target, which may comprise an eye chart or a PSF target, or a still picture of a scenery, or a live video of a scenery, or combinations thereof;
g. wherein the optics assembly is located within a line of sight of the user, and within which either two or more adjustable optics components are adjustable synchronously or individually, as directed by the Decision Module.

75. The device of claim 74, wherein the Decision Module is configured to make decisions based on eye test data and said set of rules, wherein said set of rules comprising executable codes embedded within said processor readable storage medium including:
a. A set of refraction rules built into the codes, wherein the rules are provided by one or more refraction experts, or
b. A set of refraction rules forming a decision network, that was trained to make decisions based on one of more of the following factors:
i) the Visual Acuity score at the present optics setting,
ii) the extent of improvement in Visual Acuity score between the current and the previous optics setting,
iii) a priority rule for selecting which of three optic components, sphere, cylinder and axis, to be tested next, based on the extent of Visual Acuity improvement in each of the three optical components, or
c. A combination of a) and b).

76. The device of claim 74, comprising a display or announcements of a set of instructions guiding the user through the refraction process, using voice, graphics, video, text, or combinations thereof.

77. A voice guided self refraction device as in claim 74, comprising a computer generating a voice instruction, or using a pre-recorded human voice to announce refraction instructions, wherein the voice instructions may be presented at various point of the refraction process to guide the user through the refraction process and request a response from the user, wherein the response is to indicate a choice of presented optics or to identify a Snellen letter in a Visual Acuity test.

78. The device of claim 74, comprising executable code embedded within said storage medium for recognizing the user's voice input response, using a built in voice recognition software, wherein the computer may use the user's voice input to the decision module for proceeding with the refraction process, or scoring a Visual Acuity test.

79. The device of claim 74, wherein the refraction rules are developed by refraction experts or machine learning or both, wherein input data includes:
i. The current prescription of the user, and the Visual Acuity at that prescription
ii. The auto refraction prescription and the Visual Acuity at that prescription
iii. The Visual Acuity improvement on making a change in selecting one of optical components is based on past experience
iv. Wherein the rules guide the refraction process, to attend improvement in Visual Acuity, and in a short process time.

80. The device of claim 74, comprising a self-alignment device for self-alignment of the user's eye to an auto refraction instrument, comprising;
i. An eye piece of the instrument;
ii. A light source, projecting light into the eye, light is reflected at the retina, exits the pupil, forming a lighted glow of the pupil;
iii. A camera capturing an image of the user's pupil;
iv. A monitor displaying the pupil image, wherein the user sees the pupil on the monitor;
v. A marker comprising a drawing of the boundary of a region, in which the intended eye location is within that region inside the marker's boundary, wherein the boundary can be in the
shape of a square, a circle, wherein the center of region defined by the boundary being the intended location for the eye; and
vi. Wherein the user moves the eye to the center of the boundary thereby aligning the eye to the intended position of the eye for an auto refraction image capture.

81. The device of claim 80, wherein the self-alignment device is configured to initiate capture of an eye image that is automatically triggered when the eye is inside the region defined by the marker.

82. The device of claim 80; wherein the eye image comprises wavefront data of the eye, the pupil size, and the user's papillary distance.

83.-111. (canceled)

112. A decision module configured for a self service refraction eye test instrument, comprising:
(i) code embedded within a digital storage medium for programming a processor to perform specific steps in a self-service refraction procedure; and
(ii) a database of standard refraction rules including industry standardized rules or rules provided by licensed refraction experts or both; or
(iii) a database of learned refraction rules generated by machine learning through one or both of performing or uploading data from a statistically significant number of refraction tests, or
(iv) both (ii) and (iii).

113. The decision module of claim 112, comprising:
I. rules for choosing which of the three optical components:
(a) sphere,
(b) cylinder and
(c) axis to test next;
II. rules for choosing a step size adjustment increment of a selected optical component to be tested; and
III. rules for choosing whether to continue a test, or stop the test, including determining that a final refraction result has been reached;
IV. wherein test subject visual acuity scores are input to the decision module to enable the decision process.

114. The decision module of claim 13, wherein the standard rules comprise one or more of the following:
(i) when selecting which component to test next, sphere power has a higher priority than astigmatism power which has higher priority than astigmatism axis angle, unless a magnitude of error in a lower priority component overwhelms that of a higher priority component;
(ii) when an axis angle of a right eye and a left eye are drastically non-symmetric about a 90 degree axis, then a re-testing of the axis angles is warranted;
(iii) if a spherical equivalent (SE) power of an eye under test is more minus by a certain integral multiple of 0.25D, compared with a SE power from a previous refraction result of the same eye, and an improvement in visual acuity is not increased by approximately an integral number of lines in the eye chart, then a re-test of sphere power of the eye is warranted.
